(19) United States
(12) Patent Application Publication
(10) Pub. No.: US 2009/0036687 A1
(43) Pub. Date: Feb. 5, 2009

(54) MULTINUCLEAR COMPLEX AND CONDENSATION PRODUCT THEREOF

(76) Inventors: Yoshiyuki Sugahara, Tokyo (JP); Takeshi Ishiyama, Ibaraki (JP); Hideyuki Higashimura, Ibaraki (JP)

Correspondence Address: SUGHRUE MION, PLLC, 2100 PENNSYLVANIA AVENUE, N.W., SUITE 800, WASHINGTON, DC, 20037, US

(21) Appl. No.: 12/278,415

(22) PCT Filed: Feb. 8, 2007
(86) PCT No.: PCT/JP2007/052285

§ 371 Date: Sep. 16, 2008

(30) Foreign Application Priority Data
Feb. 8, 2006 (JP) 2006-030586

(51) Int. Cl. C07D 403/14; C07F 7/10

(52) U.S. Cl. 548/106; 548/110

(57) ABSTRACT

A multinuclear complex comprising a plurality of metal atoms and a ligand L coordinating to the metal atoms, and satisfying the following conditions (i), (ii), (iii) and (iv):

(i) The ligand L has a monovalent group represented by the following general formula (1) and/or a divalent group represented by the following general formula (2),

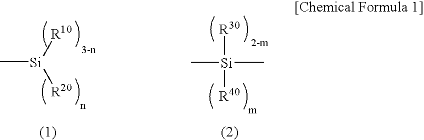

(ii) The ligand L has at least 5 coordination atoms bonding to the metal atom,
(iii) At least one of the coordination atoms bonds to two of the metal atoms, or the minimum number of covalent bonds between any two selected coordination atoms is 1-5, and
(iv) The ligand L is soluble in the solvent.

### TECHNICAL FIELD

The present invention relates to a multinuclear complex having a condensable silyl group, and to a condensate obtained by condensation of the multinuclear complex. The invention further relates to a multinuclear complex that is suitable as a redox catalyst, and to a condensate of the multinuclear complex.

### BACKGROUND ART

As defined in “Kagaku Jiten” [Dictionary of Chemistry] (1st Edition, 1994, Tokyo Kagaku Dojin), a multinuclear complex is a compound with two or more metals (ions) as central atoms in the same complex. Multinuclear complexes have specific and diverse reactivity based on interaction between the plurality of metal atoms and can therefore serve as catalysts that are capable of promoting unique reactions; they are particularly useful as catalysts for chemical reactions that involve electron transfer, such as redox catalysts (see Non-patent document 1, for example). As one type of example, dinuclear manganese complexes are used as catalysts that decompose hydrogen peroxide into water and oxygen (peroxide decomposition catalysts) while inhibiting generation of free radicals (hydroxyl radicals, hydroperoxy radicals and the like) (see Non-patent document 2, for example).

Also, multinuclear metal complexes are used not only as catalysts but also in sensors, and for example, multinuclear complexes having cryptands as macrocyclic ligands coordinating two copper ions, and converted to xerogels by sol-gel reaction, are used as azide ion detectors (see Non-patent document 3, for example).

[Non-patent document 1] K. Koyanazu, M. Yuasa, Hyomen 2003, 41(3), 22.
[Non-patent document 2] A. E. Boelrijk and G. C. Dismukes Inorg. Chem., 2000, 39, 3020.
[Non-patent document 3] Manuel G Basallote et al., Chem. Mater., 2003, 15, 2025

### DISCLOSURE OF THE INVENTION

### Problems to be Solved by the Invention

However, when the dinuclear manganese complex disclosed in Non-patent document 2 is used in a reaction system as a peroxide decomposition catalyst in the copresence of a solvent, dissolution of the catalyst can pose a problem depending on the solvent, and therefore from the viewpoint of separation and recovery of the catalyst from the reaction system and conjugation with the support carrier, it has been desirable to develop heterogeneous catalysts that are insoluble in solvents.

Moreover, xerogelled multinuclear complexes such as that of Non-patent document 3 have not been used as redox catalysts, and the present inventors have found the coordination geometry of the copper atoms in such xerogelled multinuclear complexes is insufficient for their use as redox catalysts.

It is therefore an object of the present invention to provide a multinuclear complex that not only has unique catalytic activity but also excellent thermostability, and especially to provide a heterogeneous catalyst with catalytic power capable of decomposing hydrogen peroxide to water and oxygen while inhibiting generation of free radicals, as well as a novel multinuclear complex as a precursor for the catalyst.

### Means for Solving the Problems

As a result of diligent efforts directed toward solving the problems mentioned above, the present inventors have completed this invention upon finding that a condensate or co-condensate obtained by condensation of a multinuclear complex with a specified ligand has high stability without loss of reactivity as a redox catalyst.

Specifically, the invention provides a multinuclear complex comprising a plurality of metal atoms and a ligand L coordinating to the metal atoms, and satisfying the following conditions (i), (ii), (iii) and (iv).

(i) It has a monovalent group represented by the following general formula (1) and/or a divalent group represented by the following general formula (2).

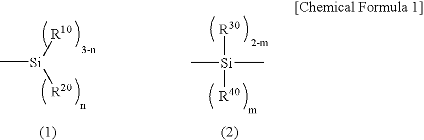

In this formula, R¹⁰ and R³⁰ represent an optionally substituted C1-10 alkyl or optionally substituted C6-10 aryl group. When more than one R¹⁰ and R³⁰ are bonded to the same Si, they may be the same or different. R²⁰ and R⁴⁰ each independently represent hydrogen, hydroxyl optionally substituted C1-10 alkoxy, optionally substituted C6-10 aryloxy, optionally substituted C2-10 acyloxy or —O—P(O)(OR⁷⁰)₂ (where R⁷⁰ represents hydrogen, or a C1-10 alkyl or C6-10 aryl group), and when more than one R²⁰ and R⁴⁰ are bonded to the same Si, they may be the same or different. The letter n represents 1, 2 or 3, and m represents 1 or 2.

(ii) The ligand L has at least 5 coordination atoms bonding to the metal atom.
(iii) At least one of the coordination atoms bonds to two of the metal atoms, or the minimum number of covalent bonds between any two selected coordination atoms is 1-5.
(iv) The ligand L is soluble in the solvent.

The multinuclear complex of the invention preferably has a nitrogen atom, oxygen atom, phosphorus atom or sulfur atom as the coordination atom of the ligand L.

Preferably, at least one of the coordination atoms in the multinuclear complex of the invention is a nitrogen atom that forms a bond represented by —C═N—.

The multinuclear complex of the invention preferably has a total of no more than 8 metal atoms in the molecule.

The metal atoms in the molecule of the multinuclear complex of the invention are preferably transition metal atoms of the first series of transition elements.

The multinuclear complex of the invention also preferably has 1 ligand L and 2 metal atoms.

The multinuclear complex of the invention also preferably has a molecular weight of no greater than 6000.

The ligand L in the multinuclear complex of the invention is preferably a compound represented by the following general formula (3).

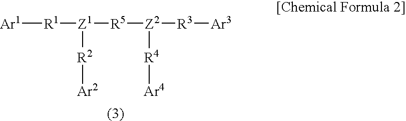

Here, Ar¹, Ar², Ar³ and Ar⁴ (hereinafter referred to as Ar¹-Ar⁴) each independently represent an aromatic heterocyclic group, R¹, R², R³, R⁴ and R⁵ (hereinafter referred to as R¹-R⁵) represent divalent groups, and Z¹ and Z² each independently represent a nitrogen atom or trivalent group. However, at least one group from among Ar¹-Ar⁴ and R¹-R⁵ contains a monovalent group represented by general formula (1) above and/or a divalent group represented by general formula (2) above.

The ligand L in the multinuclear complex of the invention is preferably a compound represented by the following general formula (4a) or (5a).

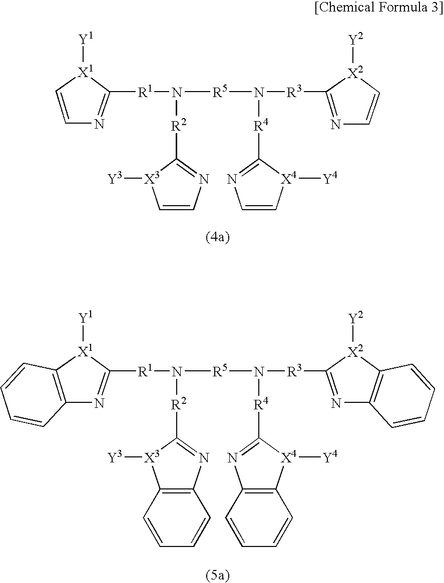

Here, R¹-R⁵ have the same definitions as above. X¹, X², X³ and X⁴ (hereinafter referred to as X¹-X⁴) each independently represent a nitrogen atom or CH group, Y¹, Y², Y³ and Y⁴ (hereinafter referred to as Y¹-Y⁴) each independently represent hydrogen, a C1-50 alkyl group or C2-60 aromatic group, or a group having the structure represented by general formula (1) or (2) above. At least one of Y¹-Y⁴ is a group having the structure represented by general formula (1) above.

The ligand L in the multinuclear complex of the invention is also preferably a compound represented by the following general formula (4b) or (5b).

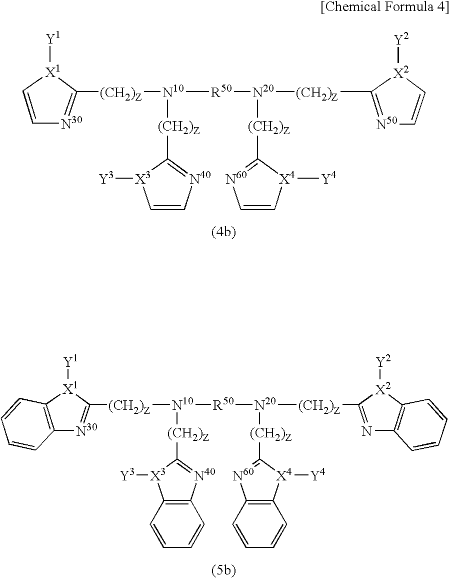

Here, X¹-X⁴ and Y¹-Y⁴ have the same definitions as above. At least one of Y¹-Y⁴ is a group having the structure represented by general formula (1) above, and Z represents 1 or 2. R⁵⁰ represents a divalent group with 2-14 covalent bonds linking N¹⁰ and N²⁰.

The ligand L in the multinuclear complex of the invention is also preferably a compound represented by the following general formula (4c) or (5c).

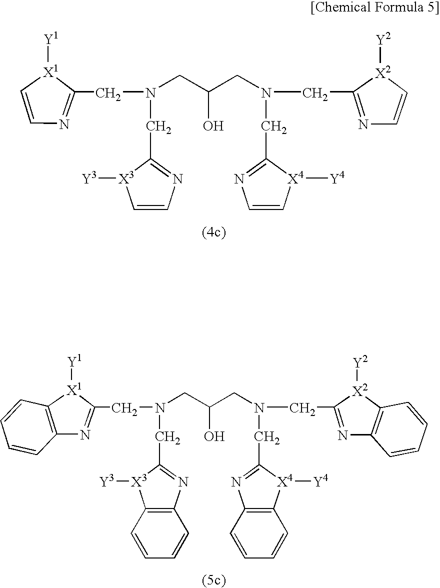

Here, X¹-X⁴ and Y¹-Y⁴ have the same definitions as above, and at least one of Y¹-Y⁴ is preferably a group having the structure represented by general formula (1) above.

The invention provides a condensate obtained by condensation of the aforementioned multinuclear complex, and the condensate is preferably obtained by condensation at a temperature of below 150° C.

The invention further provides a co-condensate obtained by co-condensation of one more of the aforementioned multinuclear complexes with a monomer capable of co-condensation with the multinuclear complexes, and the co-condensate is preferably obtained by co-condensation at a temperature of below 150° C.

The invention still further provides a redox catalyst comprising the aforementioned multinuclear complex, condensate or co-condensate.

### EFFECT OF THE INVENTION

The multinuclear complex, the condensate obtained by condensation of the multinuclear complex and the co-condensate obtained by co-condensation of the multinuclear complex, according to the invention, are useful as redox catalysts. In particular, when used for a peroxide decomposition catalyst, co-condensation with the condensate allows decomposition to water and oxygen to be accomplished while minimizing generation of free radicals, and yields a heterogeneous catalyst that is insoluble in solvents, unlike hitherto disclosed multinuclear complex catalysts. Such a heterogeneous catalyst facilitates catalyst separation and recovery from the reaction system and conjugation with materials, and can be used as an antidegradant for polymer electrolyte fuel cells and hydroelectrolysis devices or an antioxidant for medical and agricultural chemicals and food products.

### BRIEF DESCRIPTION OF THE DRAWINGS

FIG. 1 shows a ¹H-NMR spectrum for the bbpr-allyl ligand synthesized in Production Example 1.

FIG. 2 is a graph showing the time-dependent change in generated oxygen in Example 4.

FIG. 3 is a graph showing the time-dependent change in generated oxygen in Example 5.

FIG. 4 shows a ¹H-NMR spectrum for the bbpr-CH₂St ligand synthesized in Production Example 2.

### BEST MODES FOR CARRYING OUT THE INVENTION

Preferred embodiments of the invention will now be explained in detail, with reference to the accompanying drawings as necessary.

The multinuclear complex of the invention comprises a plurality of metal atoms and a ligand L coordinating to the metal atoms, and satisfying the following conditions (i), (ii), (iii) and (iv). The metal atoms may be uncharged or charged ions.

The multinuclear complex of the invention contains a plurality of metal atoms, and the number of metal atoms is preferably 2-8, more preferably 2-4 and even more preferably 2 or 3.

It also contains one or more ligands L, and the number of ligands L is preferably 1-6, more preferably 1-3, even more preferably 1 or 2 and most preferably 1.

The metal atoms in the multinuclear complex of the invention are selected from among transition metal atoms, which may be the same or different. As specific examples of transition metal atoms there may be mentioned transition metals or transition metal ions of the first series of transition elements, selected from the group consisting of scandium, titanium, vanadium, chromium, manganese, iron, cobalt, nickel, copper and zinc; as well as yttrium, zirconium, niobium, molybdenum, ruthenium, rhodium, palladium, silver, cadmium, lanthanum, cerium, praseodymium, neodymium, promethium, samarium, europium, gadolinium, terbium, dysprosium, holmium, erbium, thulium, ytterbium, lutetium, hafnium, tantalum, tungsten, rhenium, osmium, iridium, platinum, gold, mercury, actinium, thorium, protactinium and uranium.

They are preferably the aforementioned transition metal atoms of the first series of transition elements or transition metal atoms selected from the group consisting of zirconium, niobium, molybdenum, ruthenium, rhodium, palladium, silver, lanthanum, cerium, praseodymium, neodymium, promethium, samarium, europium, gadolinium, terbium, dysprosium, holmium, erbium, thulium, ytterbium, lutetium, tantalum, tungsten, rhenium, osmium, iridium, platinum and gold; more preferably transition metals or transition metal ions of the first series of transition elements or transition metals or transition metal ions selected from the group consisting of zirconium, niobium, molybdenum, ruthenium, rhodium, palladium, silver, lanthanum, cerium, samarium, europium, ytterbium, lutetium, tantalum, tungsten, rhenium, osmium, iridium, platinum and gold; even more preferably the aforementioned transition metal atoms of the first series of transition elements; most preferably vanadium, chromium, manganese, iron, cobalt, nickel or copper; and most especially transition metals or atoms selected from among manganese, iron, cobalt, nickel and copper.

The ligand L has at least one monovalent group represented by general formula (1) above and/or divalent group represented by general formula (2) above, as condition (i). The ligand L may have both of these different types of groups, and when a plurality thereof are present the groups may be either the same or different.

R¹⁰ as the monovalent group represented by general formula (1) above represents an optionally substituted C1-10 alkyl or optionally substituted C6-10 aryl group.

Examples of alkyl groups include straight-chain alkyl groups, branched alkyl groups or alkyl groups of cycloalkyl groups, such as methyl, ethyl, propyl, isopropyl, butyl, t-butyl, hexyl, cycloheptyl, cyclohexyl and the like. These alkyl groups may have substituents, and as substituents there may be mentioned hydroxyl, mercapto, sulfo, phosphono, nitro, halogeno (fluoro, chloro, bromo or iodo groups), amino and about C1-4 alkyloxy groups. When the alkyl group has an alkyloxy group as a substituent, it is selected so that the total number of carbon atoms is no greater than 10.

As C6-10 aryl groups there may be mentioned phenyl, naphthyl and biphenyl. These aryl groups may be substituted with the same substituents, about C1-4 alkyl groups or alkoxy groups, as the alkyl groups mentioned above. When the aryl group has an alkoxy or alkyl group as a substituent, it is selected so that the total number of carbon atoms is no greater than 10.

Among the examples mentioned above, R¹⁰ is preferably methyl, ethyl, butyl or phenyl.

In the formula, R²⁰ is a group selected from among hydrogen, hydroxyl, optionally substituted C1-6 alkoxy, optionally substituted C6-10 aryloxy, optionally substituted C2-6 acyloxy and groups represented by —O—P(O)(OR⁷⁰)₂ (where R⁷⁰ represents hydrogen, a C1-10 alkyl group or a C6-10 aryl group).

As C1-6 alkoxy groups there may be mentioned methoxy, ethoxy, propoxy, isopropoxy, butoxy, t-butoxy and hexyloxy, which may have the same substituents as the aforementioned alkyl groups.

As aryloxy groups there may be mentioned phenoxy, naphthoxy and biphenyloxy. The aryloxy groups may be substituted with the substituents mentioned as substituents for the aforementioned alkyl groups, or with about C1-4 alkyl groups. When the aryloxy group has an alkoxy or alkyl group as a substituent, it is selected so that the total number of carbon atoms is no greater than 10.

Examples of acyloxy groups include acetoxy, propionyloxy and butyryloxy. These may be substituted with the groups mentioned as substituents for the aforementioned alkyl groups, or with about C1-4 alkoxy or alkyl groups. When the acyloxy group has an alkoxy or alkyl group as a substituent, it is selected so that the total number of carbon atoms is no greater than 10.

Examples of groups represented by —O—P(O)(OR⁷⁰)₂ include phosphoric acid ester groups, dimethyl phosphate groups, diethyl phosphate groups and the like.

Of these examples, R²⁰ is preferably hydroxyl, methoxy, ethoxy, propoxy, phenoxy or acetoxy, and more preferably hydroxyl, methoxy or ethoxy.

In general formula (1), n is 1, 2 or 3, preferably 2 or 3 and more preferably 3.

Preferred examples of groups represented by general formula (1) above include trihydroxysilyl, trimethoxysilyl, triethoxysilyl, tripropoxysilyl, tributoxysilyl, trihexyloxysilyl, triphenoxysilyl, tritoluoyloxysilyl, trinaphthyloxysilyl, triacetoxysilyl, tripropionyloxysilyl, tributyryloxysilyl, tripivaloyloxysilyl and tribenzoyloxysilyl, with trimethoxysilyl, triethoxysilyl, tripropoxysilyl, triphenoxysilyl and triacetoxysilyl being more preferred, trimethoxysilyl, triethoxysilyl and tripropoxysilyl being even more preferred and trimethoxysilyl and triethoxysilyl being yet more preferred.

Of the groups mentioned above, a portion of the alkoxy or aryloxy groups bonding to the same Si will become hydrolyzed by moisture in the air to form silanol groups (groups wherein at least one R²⁰ is a hydroxyl group), and the preferred groups represented by general formula (1) also include such partially hydrolyzed groups.

The group represented by general formula (1) above may also have a divalent linking group as a group represented by the following formula (1a) in the ligand L.

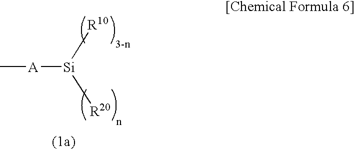

Here, R¹⁰, R²⁰ and n have the same definitions as above, and A represents a divalent organic group. As divalent organic groups represented by A there may be mentioned C1-16 alkylene, C2-60 divalent and aromatic groups (including heterocyclic aromatic groups), and these divalent groups may also be linked groups. As preferred linking groups there may be mentioned methylene, ethylene, propylene, butylene, phenylene, toluilene, (1-methyl)ethylene, (2-methyl)propylene, (2,2-dimethyl)ethylene and groups represented by the following formulas (1b), (1c), (1d) and (1e).

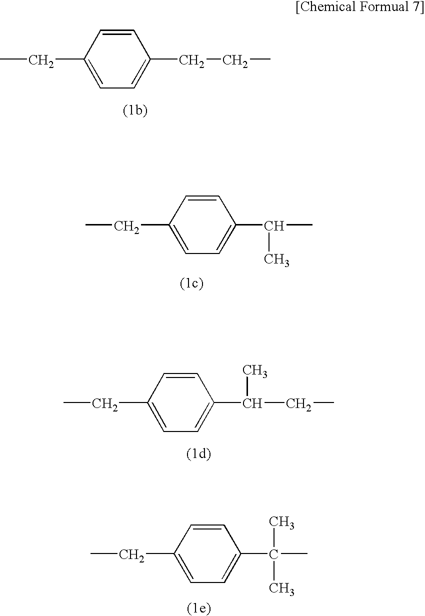

The divalent group represented by general formula (2) above will now be explained. In general formula (2), R³⁰ is a group of the same groups mentioned as R¹⁰ in general formula (1) above, and the preferred examples are also the same. R⁴⁰ is a group of the same groups mentioned as R²⁰ in general formula (1) above, and the preferred examples are also the same. In general formula (2), m is 1 or 2 and preferably 2.

As divalent groups represented by general formula (2) there are preferred dihydroxysilylene, dimethoxysilylene, diethoxysilylene, dipropoxysilylene, dibutoxysilylene, dihexyloxysilylene, diphenoxysilylene, ditoluoyloxysilylene, dinaphthyloxysilylene, diacetoxysilylene, dipropionyloxysilylene, dibutyryloxysilylene, dipivaloyloxysilylene and dibenzoyloxysilylene, with dimethoxysilylene, diethoxysilylene, dipropoxysilylene, diphenoxysilylene and diacetoxysilylene being more preferred and dimethoxysilylene and diethoxysilylene being especially preferred.

As stated under condition (ii), the ligand L has at least 5 coordination atoms that bond to the metal atom. A coordination atom is defined in “Iwasaki, Rikagaku Jiten [Dictionary of Physics and Chemistry], 4th Edition” (Ryogo Kubo, ed., Jan. 10, 1991, p. 966, Iwanami Shoten), as an atom that has an unshared electron pair that donates an electron to the unoccupied orbital of a metal atom, creating a coordination bond with the metal atom. The preferred number of coordination atoms in the ligand L is 5-20, more preferably 5-12 and even more preferably 7-10.

Any of the metal atoms in the multinuclear complex of the invention preferably has at least 3 coordination bonds with the ligand L, with 3-20 being more preferred, 3-7 being even more preferred, 4-6 being yet more preferred and 4 or 5 being especially preferred.

According to condition (iii), it is an essential condition that the ligand L has at least one of the coordination atoms bonding to two of the metal atoms, or the minimum number of covalent bonds between any two selected coordination atoms is 1-5. If a single coordination atom thus bonds to two metal atoms, the two metal atoms are crosslinked with a single coordination atom, producing “crosslinked coordination”. Also, if two coordination atoms bonding to different metal atoms are represented as AM1 and AM2, the metal atom in a coordination bond with AM1 is represented as M¹ and the metal atom in a coordination bond with AM2 is represented as M², then M¹ and M² will be positioned in mutual proximity in the multinuclear metal complex molecule and superior catalytic activity can be obtained. The combination is preferably such that the minimum number of covalent bonds between AM1-AM2 is no greater than 4, more preferably no greater than 3, even more preferably no greater than 2 and most preferably no greater than 1. The ligand L most preferably has a coordination atom that can form a crosslinked coordination structure by coordinating two metal atoms with a single coordination atom.

The coordination atom is preferably an atom selected from among carbon atoms, nitrogen atoms, oxygen atoms, phosphorus atoms and sulfur atoms, more preferably nitrogen atoms, oxygen atoms, phosphorus atoms and sulfur atoms, even more preferably nitrogen atoms, oxygen atoms and sulfur atoms and most preferably nitrogen atoms and oxygen atoms. The plurality of coordination atoms may be the same or different.

As specified by condition (iv) above, the ligand L itself, i.e. the compound that serves as the ligand L, must be soluble in the solvent. There are no particular restrictions on the solvent, but it is preferably a solvent that allows the complex-forming reaction to proceed smoothly to facilitate production of the multinuclear complex.

Preferably, at least one of the coordination atoms among the coordination atoms in the ligand L is a nitrogen atom that forms a bond represented by —C═N—. Such a nitrogen atom is preferably included as a coordination atom for more excellent redox catalytic activity and especially catalytic activity in peroxide decomposition reactions. The nitrogen atom in a carbon-nitrogen double bond may be the nitrogen atom of an imino group obtained by condensation of the carbonyl group of a ketone compound or aldehyde compound with an amine compound, or the nitrogen atom of an aromatic heterocyclic ring with a carbon-nitrogen double bond.

If the ligand L has an aromatic heterocyclic ring with a carbon-nitrogen double bond, this means that the ligand L contains a monovalent or greater aromatic heterocyclic group derived by removing one or more hydrogens from an aromatic heterocyclic molecule or a fused ring molecule containing an aromatic heterocyclic molecule. The aromatic heterocyclic group may also have a substituent.

Examples of such aromatic heterocyclic molecules include imidazole, pyrazole, 2H-1,2,3-triazole, 1H-1,2,4-triazole, 4H-1,2,4-triazole, 1H-tetrazole, oxazole, isooxazole, thiazole, isothiazole, furazan, pyridine, pyrazine, pyrimidine, pyridazine, 1,3,5-triazine and 1,3,4,5-tetrazine.

Examples of the aforementioned fused ring molecules include benzimidazole, 1H-indazole, benzoxazole, benzothiazole, quinoline, isoquinoline, cinnoline, quinazoline, quinoxaline, phthalazine, 1,8-naphthylidine, pteridine, phenanthridine, 1,10-phenanthroline, purine, pteridine and perimidine.

Of the aromatic heterocyclic groups mentioned above, there are preferred monovalent or greater aromatic heterocyclic groups derived by removing one or more hydrogens from an aromatic heterocyclic molecule or fused ring molecule such as imidazole, pyrazole, pyridine, pyrazine, pyrimidine, pyridazine, benzimidazole, 1H-indazole, quinoline, isoquinoline, cinnoline, phthalazine, 1,8-naphthylidine or purine.

The aromatic heterocyclic molecule or fused ring molecule may also contain a monovalent substituent, examples of which include hydroxyl, mercapto, carboxyl, phosphono, sulfo, nitro, halogeno (fluoro, chloro, bromo or iodo groups), carbamoyl, C1-50 alkyl, C2-60 aromatic groups (including aromatic heterocyclic groups), alkoxy or alkylthio groups comprising the aforementioned alkyl groups and ether or thioether groups, aryloxy or arylthio groups comprising the aforementioned aromatic groups and ether or thioether groups, alkylsulfonyl or arylsulfonyl groups comprising the aforementioned alkyl or aromatic groups and sulfonyl groups, acyl or arylcarbonyl groups comprising the aforementioned alkyl or aromatic groups and carbonyl groups, alkyloxycarbonyl or aryloxycarbonyl groups comprising the aforementioned alkyl or aromatic groups and oxycarbonyl groups, amino groups optionally having one of the aforementioned alkyl or aromatic groups, acid amide groups optionally having one of the aforementioned alkyl or aromatic groups, phosphoryl groups optionally having one of the aforementioned alkyl and/or aromatic groups, thiophosphoryl groups optionally having one of the aforementioned alkyl and/or aromatic groups, and silyl groups optionally having one of the aforementioned alkyl and/or aromatic groups.

As examples of C1-50 alkyl groups there may be mentioned alkyl groups derived by removing one hydrogen from a saturated hydrocarbon compound, such as a straight-chain alkyl, branched alkyl or cycloalkyl group such as methyl, ethyl, propyl, isopropyl, butyl, isobutyl, 2,2-dimethylbutyl, octyl, decyl, dodecyl, hexadecyl, eicosyl, triacontyl, pentacontyl, cyclopentyl, cyclohexyl or adamantyl.

Such alkyl groups are preferably C1-30 alkyl groups, more preferably C1-16 alkyl groups and most preferably C1-8 alkyl groups.

As examples of such aromatic groups (including aromatic heterocyclic groups) there may be mentioned aromatic groups derived by removing one hydrogen from about C2-60 aromatic compounds (including aromatic ring heterocyclic compounds) such as phenyl, toluoyl, 4-t-butylphenyl, naphthyl, furyl, thiophenyl, pyrroyl, pyridyl, furazanyl, oxazoyl, imidazoyl, pyrazoyl, pyrazyl, pyrimidyl, pyridazyl, benzimidazoyl and triazinyl.

As such aromatic groups there are preferred C1-30 aromatic groups, more preferably C1-16 aromatic groups and even more preferably C1-8 aromatic groups.

The aforementioned saturated hydrocarbon compounds or aromatic compounds may also having hydroxyl, mercapto, carboxyl, sulfo, phosphono, nitro, halogeno or silyl groups (where the silyl groups have three groups selected from among C1-50 alkyl and C2-60 aromatic groups), as well as monovalent groups represented by general formula (1) above and groups represented by general formula (2) above.

The multinuclear complex of the invention may also have another ligand in addition to the aforementioned ligand L. The other ligands may be ionic or electrically neutral compounds, and when a plurality of other ligands are present, the ligands may be the same or different.

Examples of electrically neutral compounds for other ligands include nitrogen atom-containing compounds such as ammonia, pyridine, pyrrole, pyridazine, pyrimidine, pyrazine, 1,2,4-triazine, pyrazole, imidazole, 1,2,3-triazole, oxazole, isooxazole, 1,3,4-oxadiazole, thiazole, isothiazole, indole, indazole, quinoline, isoquinoline, phenanthridine, cinnoline, phthalazine, quinazoline, quinoxaline, 1,8-naphthylidine, acridine, 2,2′-bipyridine, 4,4′-bipyridine, 1,10-phenanthroline, ethylenediamine, propylenediamine, phenylenediamine, cyclohexanediamine, pyridine N-oxide, 2,2′-bipyridine N,N′-dioxide, oxamide, dimethylglyoxime and o-aminophenol; oxygen-containing compounds such as water, phenol, oxalic acid, catechol, salicylic acid, phthalic acid, 2,4-pentanedione, 1,1,1-trifluoro-2,4-pentanedione, hexafluoropentanedione, 1,3-diphenyl-1,3-propanedione and 2,2′-binaphthol; sulfur-containing compounds such as dimethyl sulfoxide and urea; and phosphorus-containing compounds such as 1,2-bis(dimethylphosphino)ethane and 1,2-phenylenebis(dimethylphosphine). Preferred examples are ammonia, pyridine, pyrrole, pyridazine, pyrimidine, pyrazine, 1,2,4-triazine, pyrazole, imidazole, 1,2,3-triazole, oxazole, isooxazole, 1,3,4-oxadiazole, indole, indazole, quinoline, isoquinoline, phenanthridine, cinnoline, phthalazine, quinazoline, quinoxaline, 1,8-naphthylidine, acridine, 2,2′-bipyridine, 4,4′-bipyridine, 1,10-phenanthroline, ethylenediamine, propylenediamine, phenylenediamine, cyclohexanediamine, pyridine N-oxide, 2,2′-bipyridine N,N′-dioxide, oxamide, dimethylglyoxime, o-aminophenol, water, phenol, oxalic acid, catechol, salicylic acid, phthalic acid, 2,4-pentanedione, 1,1,1-trifluoro-2,4-pentanedione, hexafluoropentanedione, 1,3-diphenyl-1,3-propanedione and 2,2′-binaphthol, and more preferred examples are ammonia, pyridine, pyrrole, pyridazine, pyrimidine, pyrazine, 1,2,4-triazine, pyrazole, imidazole, 1,2,3-triazole, oxazole, isooxazole, 1,3,4-oxadiazole, indole, indazole, quinoline, isoquinoline, phenanthridine, cinnoline, phthalazine, quinazoline, quinoxaline, 1,8-naphthylidine, acridine, 2,2′-bipyridine, 4,4′-bipyridine, 1,10-phenanthroline, ethylenediamine, propylenediamine, phenylenediamine, cyclohexanediamine, pyridine N-oxide, 2,2′-bipyridine N,N′-dioxide, o-aminophenol, phenol, catechol, salicylic acid, phthalic acid, 1,3-diphenyl-1,3-propanedione and 2,2′-binaphthol.

Preferred as other ligands that are electrically neutral among those mentioned above are pyridine, pyrrole, pyridazine, pyrimidine, pyrazine, pyrazole, imidazole, oxazole, indole, quinoline, isoquinoline, acridine, 2,2′-bipyridine, 4,4′-bipyridine, 1,10-phenanthroline, phenylenediamine, pyridine N-oxide, 2,2′-bipyridine N,N′-dioxide, o-aminophenol and phenol.

As anionic ligands there may be mentioned hydroxide ion, peroxide, superoxide, cyanide ion, thiocyanate ion, halide ions such as fluoride ion, chloride ion, bromide ion and iodide ion, sulfate ion, nitrate ion, carbonate ion, perchlorate ion, tetraarylborate ions such as tetrafluoroborate ion and tetraphenylborate ion, hexafluorophosphate ion, methanesulfonate ion, trifluoromethanesulfonate ion, p-toluenesulfonate ion, benzenesulfonate ion, phosphate ion, phosphite ion, acetate ion, trifluoroacetate ion, propionate ion, benzoate ion, hydroxide ion, metal oxide ions, methoxide ion and ethoxide ion. Preferred are hydroxide ion, sulfate ion, nitrate ion, carbonate ion, perchlorate ion, tetrafluoroborate ion, tetraphenylborate ion, hexafluorophosphate ion, methanesulfonate ion, trifluoromethanesulfonate ion, p-toluenesulfonate ion, benzenesulfonate ion, phosphate ion, acetate ion, trifluoroacetate ion and hydroxide ion. More preferred among those mentioned above are hydroxide ion, sulfate ion, nitrate ion, carbonate ion, tetraphenylborate ion, trifluoromethanesulfonate ion, p-toluenesulfonate ion, acetate ion and trifluoroacetate ion.

The ions mentioned above as examples of anionic ligands may also function as counter ions for electrical neutralization of the multinuclear metal complex of the invention itself.

The multinuclear complex of the invention will sometimes have a cationic counter ion for electrical neutrality. Examples of cationic counter ions include alkali metal ions, alkaline earth metal ions, tetraalkylammonium ions such as tetra(n-butyl)ammonium ion and tetraethylammonium ion, and tetraarylphosphonium ions such as tetraphenylphosphonium ion, and specifically there may be mentioned lithium ion, sodium ion, potassium ion, rubidium ion, cesium ion, magnesium ion, calcium ion, strontium ion, barium ion, tetra(n-butyl)ammonium ion, tetraethylammonium ion and tetraphenylphosphonium ion, and more preferably tetra(n-butyl)ammonium ion, tetraethylammonium ion and tetraphenylphosphonium ion.

Of these, tetra(n-butyl)ammonium ion and tetraethylammonium ion are preferred as cationic counter ions. By appropriately selecting the counter ion used, it is possible to adjust the solubility and dispersibility of the multinuclear complex in the solvent.

Also, the multinuclear complex of the invention preferably has a molecular weight of no greater than 6000. A molecular weight within this range is preferred in order to facilitate synthesis of the multinuclear complex itself. The molecular weight is more preferably no greater than 5000, even more preferably no greater than 4000 and most preferably no greater than 2000. There are no particular restrictions on the lower limit of the molecular weight of the multinuclear complex, but it may be about 230. A lower molecular weight is preferred for the multinuclear complex for greater convenience of operation during condensation or co-condensation of the multinuclear complex, as explained hereunder.

Suitable compounds for the ligand L in the multinuclear complex of the invention will now be described. As mentioned above, the ligand L preferably contains a nitrogen atom that forms a bond represented by —C═N— as a coordination atom, and more preferably it contains a nitrogen atom that forms a bond represented by —C═N— in an aromatic heterocyclic group.

Examples for the ligand L having a nitrogen atom that forms a bond represented by —C═N— include compounds obtained by substituting hydrogens of compounds described in the literature (Anna L. Gavrilova and Brice Bosnich, Chem. Rev. 2004, 104, 349), namely Ligand Numbers 52-55, 56a, 56b, 56c, 57a, 57b, 57c, 57d, 58a, 58b, 58c and 60 in Table 5 (p. 357); Ligand Numbers 73 and 74 in Table 7 (p. 360); Ligand Numbers 79, 80, 83 and 85 in Table 8 (p. 362); Ligand Numbers 90, 91 and 92 in Table 9 (p. 364); Ligand Numbers 100-111 and 113-118 in Table 10 (p. 366); Ligand Numbers 123-132, 134-138 and 141-147; in Table 11 (p. 370-371); Ligand Numbers 151, 152 and 154-157 in Table 12 (p. 373); Ligand Numbers 166 and 167 in Table 13 (p. 376); Ligand Number 174 in Table 14 (p. 377); and Ligand Number 177 in Table 15 (p. 378), with the monovalent groups represented by general formula (1) above or the monovalent groups represented by general formula (1a) above, or the aforementioned compounds containing divalent groups represented by general formula (2) above.

Most preferred for the ligand L among the examples mentioned above are those with aromatic heterocyclic groups containing carbon-nitrogen double bonds, of which examples include compounds obtained by substituting hydrogens of compounds described in the aforementioned publication, namely Ligand Numbers 52-55, 56a, 56b, 56c, 57a, 57b, 57c, 57d, 58a, 58b, 58c and 60 in Table 5 (p. 357); Ligand Numbers 73 and 74 in Table 7 (p. 360); Ligand Numbers 79, 80, 83 and 85 in Table 8 (p. 362); Ligand Numbers 90, 91 and 92 in Table 9 (p. 364); Ligand Numbers 100, 101, 106-108, 110, 111 and 113-118 in Table 10 (p. 366); Ligand Numbers 123, 124, 126, 129, 131, 132, 134-138 and 141-147 in Table 11 (p. 370-371); Ligand Numbers 155-157 in Table 12 (p. 373); Ligand Number 174 in Table 14 (p. 377) and Ligand Numbers 177 and 179 in Table 15 (p. 378), with the monovalent groups represented by general formula (1) above or the monovalent groups represented by general formula (1a) above, or the aforementioned compounds containing divalent groups represented by general formula (2) above.

The ligand L in the multinuclear complex of the invention preferably has an aromatic heterocyclic group and a molecular weight of no greater than 6000, and compounds represented by the following general formula (3) are particularly preferred from both of these viewpoints.

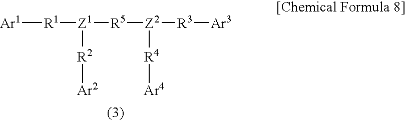

Here, Ar¹, Ar², Ar³ and Ar⁴ (hereinafter referred to as Ar¹-Ar⁴) each independently represent an aromatic heterocyclic group, R¹, R², R³, R⁴ and R⁵ (hereinafter referred to as R¹-R⁴) represent divalent linking groups, and Z¹ and Z² each independently represent a nitrogen atom or trivalent group. At least one of Ar¹-Ar⁴ and R¹-R⁵ has a group represented by general formula (1) above and/or a group represented by general formula (2).

Here, Ar¹-Ar⁴ is preferably the aforementioned aromatic heterocyclic group, examples of which include imidazolyl, pyrazolyl, 2H-1,2,3-triazolyl, 1H-1,2,4-triazolyl, 4H-1,2,4-triazolyl, 1H-tetrazolyl, oxazolyl, isooxazolyl, thiazolyl, isothiazolyl, furazyl, pyridyl, pyrazyl, pyrimidyl, pyridazyl, 1,3,5-triazilyl, 1,3,4,5-tetrazilyl, benzimidazoyl, 1H-indazoyl, benzoxazoyl, benzothiazoyl, quinolyl, isoquinolyl, cinnolyl, quinazoyl, quinoxalyl, phthalazyl, 1,8-naphthylidyl, pteridyl, carbazolyl, phenanthridyl, 1,10-phenanthrolyl, puryl, pteridyl and perimidyl.

These aromatic heterocyclic groups may also have substituents. Examples of substituents include the same substituents mentioned for the aromatic heterocyclic molecule or fused ring molecule described above. Any combination of substitution position and number of the substituents may be selected. The aromatic heterocyclic group may be bonded with a group represented by general formula (1) or general formula (1a) above, and may also have a divalent group represented by general formula (2) above.

As aromatic heterocyclic groups Ar¹-Ar⁴ in general formula (3) there are preferred benzimidazoyl, pyridyl, imidazoyl, pyrazoyl, oxazoyl, thiazolyl, isooxazolyl, isothiazolyl, pyrazyl, pyrimidyl, pyridazyl, and N-alkylbenzimidazoyl or N-alkylimidazoyl having the aforementioned alkyl groups on nitrogen atoms, there are more preferred benzimidazoyl, pyridyl, imidazoyl, pyrazoyl, pyrazyl, pyrimidyl, pyridazyl, N-alkylbenzimidazoyl and N-alkylimidazoyl, there are even more preferred benzimidazoyl, N-alkylbenzimidazoyl, pyridyl, imidazoyl, N-alkylimidazoyl and pyrazoyl, and there are most preferred pyridyl, N-alkylbenzimidazoyl and N-alkylimidazoyl.

R⁵ is a divalent group optionally having a coordination atom or coordination atom-containing group and is selected from among the alkylene groups, divalent aromatic groups and divalent heteroatom-containing groups mentioned below, or it is a combination of any of these groups linked together.

As examples of alkylene groups there may be mentioned alkylene groups obtained by removing two hydrogens from a saturated hydrocarbon molecule with a total of about 1-50 carbon atoms, such as methane, ethane, propane, butane, octane, decane, eicosane, triacontane, pentacontane, cycloheptane, cyclohexane or adamantane.

These alkylene groups may also have substituents at any desired position, with any desired number and combination of substituents, and as substituents there may be mentioned the same ones as for the aromatic heterocyclic molecules and fused ring molecules.

As alkylene groups there are preferred C1-30, more preferably C1-16, even more preferably C1-8 and most preferably C1-4 alkylene groups.

As examples of the aforementioned divalent aromatic groups there may be mentioned groups derived by removing two hydrogens from aromatic compounds and heterocyclic compounds such as benzene, naphthalene, anthracene, tetracene, biphenyl, acenaphthylene, phenalene, pyrene, furan, thiophene, pyrrole, pyridine, oxazole, isooxazole, thiazole, isothiazole, imidazole, pyrazole, pyrazine, pyrimidine, pyridazine, benzofuran, isobenzofuran, 1-benzothiophene, 2-benzothiophene, indole, isoindole, indolizine, carbazole, xanthene, quinoline, isoquinoline, 4H-quinolysine, phenanthridine, acridine, 1,8-naphthylidine, benzimidazole, 1H-indazole, quinoxaline, quinazoline, cinnoline, phthalazine, purine, pteridine, perimidine, 1,10-phenanthroline, thianthrene, phenoxathine, phenoxazine, phenothiazine, phenazine and phenarsazine, as well as these compounds with substituents.

Among the above there are preferred groups derived by removing two hydrogens from compounds selected from among benzene, phenol, p-cresol, naphthalene, biphenyl, furan, thiophene, pyrrole, pyridine, oxazole, isooxazole, thiazole, isothiazole, imidazole, pyrazole, pyrazine, pyrimidine, pyridazine, benzofuran, isobenzofuran, 1-benzothiophene, 2-benzothiophene, indole, isoindole, indolizine, carbazole, xanthene, quinoline, isoquinoline, 1,8-naphthylidine, benzimidazole, 1H-indazole, quinoxaline, quinazoline, cinnoline, phthalazine, purine, pteridine and perimidine, more preferably groups derived by removing two hydrogens from compounds selected from among benzene, naphthalene, biphenyl, pyrrole, pyridine, oxazole, isooxazole, thiazole, isothiazole, imidazole, pyrazole, pyrazine, pyrimidine, pyridazine, indole, isoindole, quinoline, isoquinoline, 1,8-naphthylidine, benzimidazole, 1H-indazole, quinoxaline, quinazoline, cinnoline and phthalazine, even more preferably groups derived by removing two hydrogens from compounds selected from among benzene, phenol, p-cresol, naphthalene, biphenyl, pyrrole, pyridine, imidazole, pyrazole, pyrazine, pyridazine, indole, isoindole, quinoline, isoquinoline, 1,8-naphthylidine, benzimidazole, 1H-indazole, quinoxaline, quinazoline, cinnoline and phthalazine, and most preferably groups derived by removing two hydrogens from compounds selected from among phenol, p-cresol, pyridine, pyrazole, pyridazine, 1,8-naphthylidine, 1H-indazole and phthalazine.

These divalent aromatic groups may also have substituents at any desired position, with any desired number and combination of substituents, and as substituents there may be mentioned the same substituents as above for the aromatic heterocyclic molecules and fused ring molecules.

As examples of divalent heteroatom-containing groups there may be mentioned groups represented by the following formulas (E-1)-(E-10).

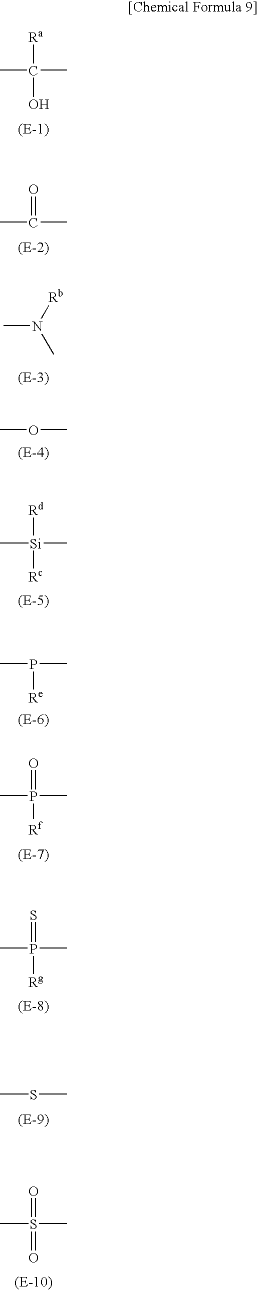

Here, R^{a}, R^{e}, R^{f} and R^{g} represent C1-50 alkyl, C2-60 aromatic, C1-50 alkoxy, C2-60 aryloxy or hydroxyl groups, or hydrogen. R^{b} represents a C1-50 alkyl group, C2-60 aromatic group or hydrogen, and R^{d} and R^{c} represent C1-50 alkyl or C2-60 aromatic groups.

Of these, groups represented by formulas (E-1), (E-2), (E-3), (E-4), (E-5), (E-7), (E-8) and (E-10) are preferred, groups represented by (E-1), (E-2), (E-4), (E-7) and (E-10) are more preferred, and groups represented by (E-1) and (E-7) are even more preferred.

R⁵ preferably contains a coordination atom. As examples of functional groups containing coordination atoms there may be mentioned hydroxyl, carboxyl, mercapto, sulfo, phosphono, nitro, cyano, ether, acyl, ester, amino group, carbamoyl, acid amide groups, phosphoryl, thiophosphoryl, sulfide, sulfonyl, pyrolyl, pyridyl, oxazolyl, isooxazolyl, thiazolyl, isothiazolyl, imidazolyl, pyrazolyl, pyrazyl, pyrimidyl, pyridazyl, indolyl, isoindolyl, carbazolyl, quinolyl, isoquinolyl, 1,8-naphthylidyl, benzimidazolyl, 1H-indazolyl, quinoxalyl, quinazolyl, cinnolyl, phthalazyl, puryl, pteridyl and perimidyl. Among these there are preferred hydroxyl, carboxyl, sulfo, phosphono, nitro, cyano, ether, acyl, amino group, phosphoryl, thiophosphoryl, sulfonyl, pyrolyl, pyridyl, oxazolyl, isooxazolyl, thiazolyl, isothiazolyl, imidazolyl, pyrazolyl, pyrazyl, pyrimidyl, pyridazyl, indolyl, isoindolyl, quinolyl, isoquinolyl, 1,8-naphthylidyl, benzimidazolyl, 1H-indazolyl, quinoxalyl, quinazolyl, cinnolyl, phthalazyl, puryl, pteridyl and perimidyl, and there are more preferred hydroxyl, carboxyl, sulfo, phosphono, cyano, ether, acyl, amino group, phosphoryl, sulfonyl, pyridyl, imidazolyl, pyrazolyl, pyrimidyl, pyridazyl, quinolyl, isoquinolyl, 1,8-naphthylidyl, benzimidazolyl, 1H-indazolyl, cinnolyl, phthalazyl and pteridyl. R⁵ is preferably a group represented by the following formula (R5-1), (R5-2), (R5-3) or (R5-4), and most preferably a group represented by the following formula (R5-1).

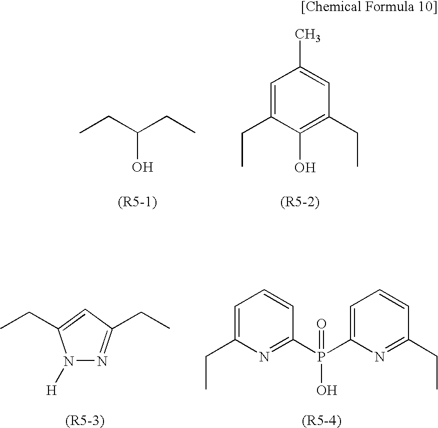

Here, the hydroxyl group in the group represented by formula (R5-1) or (R5-2), the pyrazole ring in the group represented by formula (R5-3) and the hydroxyphosphoryl group in the group represented by formula (R5-4) may release a proton to become anionic, when it is coordinated as a ligand with the metal atom.

In general formula (3), R¹-R⁴ are optionally substituted divalent groups, and they may be the same or different. As examples of R¹-R⁴ there may be mentioned the same alkylene groups, divalent aromatic groups and divalent heteroatom-containing organic groups which were mentioned for R⁵, and combinations of three of these groups in any desired linkage. R¹-R⁴ are preferably methylene, 1,1-ethylene, 2,2-propylene, 1,2-ethylene or 1,2-phenylene groups, and more preferably methylene or 1,2-ethylene groups.

Z¹ and Z² in general formula (3) are selected from among nitrogen atoms and trivalent organic groups, and the following may be mentioned as examples of trivalent organic groups.

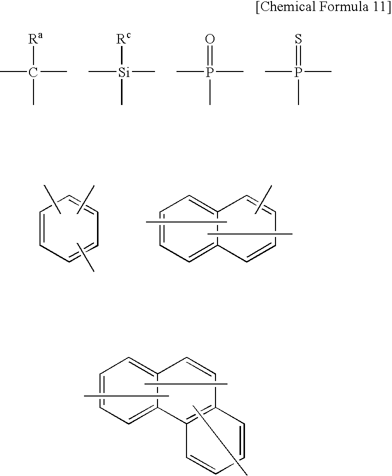

Here, R^{a} and R^{c} have the same definitions as above.

One of Z¹ and Z² is preferably a nitrogen atom, and most preferably both are nitrogen atoms. Specifically, the ligand L represented by general formula (3) above is preferably a compound represented by the following formula (4).

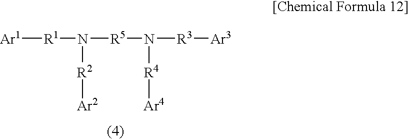

Ar¹-Ar⁴ and R¹-R⁵ have the same definitions as above, and at least one thereof has a monovalent group represented by general formula (1) above and/or a divalent group represented by general formula (2) above.

The ligand L is more preferably a compound represented by the following general formula (4a) or (5a), among the compounds represented by general formula (4) above.

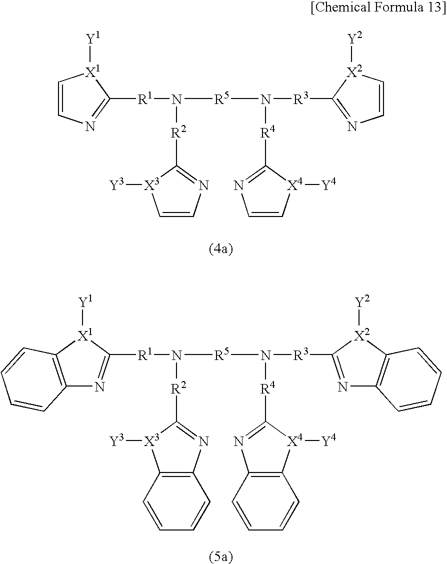

Here, R¹-R⁵ have the same definitions as above and X¹-X⁴ represent nitrogen atoms or CH. Y¹-Y⁴ represents hydrogen, C1-50 alkyl, C2-60 aromatic or a group having the structure of general formula (1) or (2), and at least one of Y¹-Y⁴ is a group having the structure of general formula (1) or (2).

A group containing a group represented by general formula (1) for Y¹-Y⁴ in general formula (4a) or (5a) is either the group represented by general formula (1) itself, or it is a monovalent group containing a group represented by general formula (1), which may be a group represented by general formula (1a) above.

Of the compounds represented by general formula (4a) or (5a), compounds represented by the following general formula (4b) or (5b) are most preferred to facilitate production.

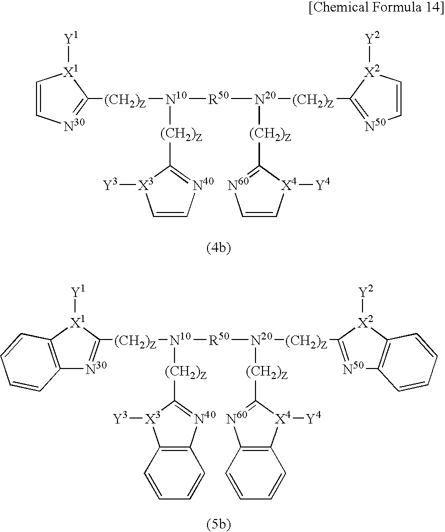

Here, X¹-X⁴ and Y¹-Y⁴ have the same definitions as above. At least one of Y¹-Y⁴ is a group containing a group represented by general formula (1) above. Z represents an integer of 1 or 2. N¹⁰ and N²⁰ represent nitrogen atoms bonded to R⁵⁰, and N³⁰, N⁴⁰, N⁵⁰ and N⁶⁰ represent nitrogen atoms in an aromatic heterocyclic group. R⁵⁰ represents a divalent group with 2-14 covalent bonds linking N¹⁰ and N²⁰. A group containing a group represented by general formula (1) for Y¹-Y⁴ is either the group represented by general formula (1) itself, or it is a monovalent group containing a group represented by general formula (1), which may be a group represented by general formula (1a) above.

Of the compounds represented by general formula (4b) or (5b), compounds represented by the following general formula (4c) or (5c) are most preferred in order to form a stable complex.

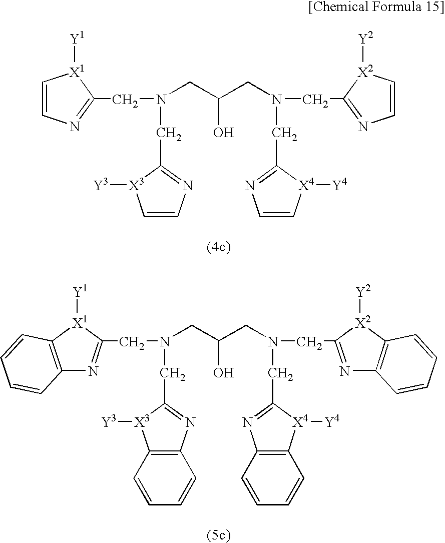

Here, X¹-X⁴ and Y¹-Y⁴ have the same definitions as above, and at least one of Y¹-Y⁴ is a group containing a group represented by general formula (1) above.

At least one of Y¹-Y⁴ in the compound represented by general formula (4c) or (5c) is a group containing a group represented by general formula (1). Preferably, two or more of Y¹-Y⁴ are groups containing a group represented by general formula (1) above, more preferably two or more of Y¹-Y⁴ are groups containing a group represented by general formula (1) above, and most preferably all of Y¹-Y⁴ are groups containing a group represented by general formula (1) above.

The method of synthesizing the multinuclear complex having the aforementioned preferred compounds as ligands L may be any of various methods. The following two main methods may be mentioned as examples.

As a first example, there may be mentioned a synthesis method in which a multinuclear complex is first synthesized having a group with a carbon-carbon double bond or carbon-carbon triple bond, and then the carbon-carbon double bond portion or carbon-carbon triple bond portion of the multinuclear complex is hydrosilylated with a hydrosilane to introduce a group represented by formula (1) or (2) above into the complex. As specific examples there may be mentioned the synthesis methods for Mn-(bbpr-SiOR)—OTf, Mn-vb-(bbpr-CH₂StSiOR)-vbSiOR, Co-(bbpr-CH₂StSiOR)—BPh₄, Ni-(bbpr-CH₂StSiOR)—BPh₄, Cu-(bbpr-CH₂StSiOR)—BPh₄ and Fe-(bbpr-CH₂StSiOR)—BPh₄ described in the examples provided below.

As a second example there may be mentioned a method in which a compound that supplies the ligand L is mixed with a transition metal compound in a solvent. The compound that supplies the ligand L may be a compound represented by the precursor compound of the ligand L or the ligand compound, i.e. the structure of the ligand L itself. The transition metal compound is preferably one that is soluble in the solvent. As preferred ligands L there may be mentioned those listed above as examples. As preferred transition metal compounds there may be mentioned transition metal salts that are soluble in the solvent. As preferred transition metal atoms in the transition metal salt there may be mentioned any of those listed above as examples. By adding an appropriate salt during the complex-forming reaction, it is possible to convert the counter ion in the complex catalyst to one from the added salt. Preferred added salts are those containing the aforementioned preferred counter ions.

As hydrosilanes to be used for the hydrosilylation reaction there may be mentioned trichlorosilane and hydrosilanes represented by the following general formula (100). When trichlorosilane is used, the product of the hydrosilylation reaction may be subjected to alcoholysis or hydrolysis to introduce a group represented by general formula (1) above into the complex.

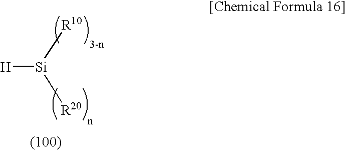

Here, R10, R²⁰ and n have the same definitions as above. The letter n is preferably 2 or 3 and more preferably 3.

As examples of hydrosilanes represented by general formula (100) above there may be mentioned trimethoxysilane, triethoxysilane, tripropoxysilane, tributoxysilane, trihexyloxysilane, triphenoxysilane, tritoluoyloxysilane, trinaphthyloxysilane, triacetoxysilane, tripropionyloxysilane, tributyryloxysilane, tripivaloyloxysilane and tribenzoyloxysilane. Among these, trimethoxysilane, triethoxysilane, tripropoxysilane, triphenoxysilane and triacetoxysilane are preferred, trimethoxysilane, triethoxysilane and tripropoxysilane are more preferred and trimethoxysilane and triethoxysilane are even more preferred.

A portion of alkoxy or aryloxy groups bonded to the same Si atom in the hydrosilane will sometimes be hydrolyzed by moisture in the air, forming silanol groups. Preferred groups represented by general formula (100) above include such partially hydrolyzed groups.

In the hydrosilylation reaction, it is preferred to use a catalyst that promotes the reaction. As catalysts there may be mentioned hexachloroplatinic (IV) acid and Karstedt catalysts, which are described in the literature (Yuki Gosei no tame no Shokubai Hannou [Catalyst Reactions For Organic Synthesis] 103, 1st Edition, 1st Printing, T. Hinokiyama, K. Nozaki, Tokyo Kagaku Dojin, p. 114-115).

The hydrosilylation reaction may be carried out in air as the reaction atmosphere, or it is preferably carried out in an inert atmosphere such as nitrogen gas or argon gas. The hydrosilylation may also be carried out either with or without a solvent. When a solvent is used, the solvent is preferably used after dehydration treatment. As such solvents there may be mentioned tetrahydrofuran, ether, 1,2-dimethoxyethane, acetonitrile, benzonitrile, 1-methyl-2-pyrrolidinone, dimethylformamide, dimethyl sulfoxide, hexane, pentane, benzene, toluene and xylene. These solvents may be used alone or in combinations of two or more.

As preferred multinuclear complexes with two metal atoms there may be mentioned complexes represented by the following general formula (6), for example, based on the aforecited literature (Ligand Number 110 (Table 10 (p. 366), Anna L. Gavrilova and Brice Bosnich Chem. Rev. 2004, 104, 349).

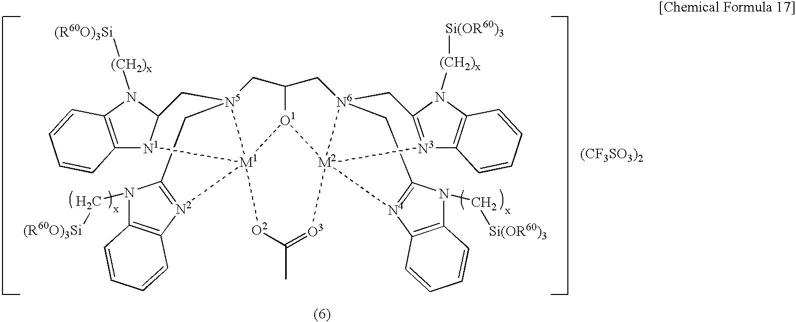

Here, the coordination atom-containing aromatic heterocyclic groups (Ar¹-Ar⁴) in the ligand L have four benzimidazolyl groups, with M¹ or M² (the dotted lines connecting M¹ and M² represent coordination bonds) coordinated with one nitrogen atom of each benzimidazolyl group as the coordination atom (represented as N¹, N², N³ and N⁴), and silylalkyl groups with polymerization reactivity bonded to the other nitrogen atom of each benzimidazolyl group. The linking groups represented by R¹-R⁴ have methylene, and R⁵ has a trimethylene group with an alcoholate group as the crosslinked coordination atom (represented by O¹). The multinuclear complex has an acetate ion (having O² and O³ as coordination atoms) as a ligand other than the ligands L and two trifluoromethane sulfonate ion molecules as a counter ion. The letter x represents 2 or 3, and R⁶⁰ represents methyl, ethyl, propyl, butyl or phenyl.

The superscript numerals on the nitrogen coordination atoms and oxygen coordination atoms are for reference in the explanation provided below regarding the number of covalent bonds between the coordination atoms.

The number of covalent bonds between coordination atoms bonding to M¹ and M² in the complex represented by general formula (6) will now be explained. In the complex of general formula (6), M¹ and M² are coordinated to the same coordination atom O¹ at M¹-O¹-M², the minimum number of covalent bonds linking the coordination atoms at M¹-O²—O³-M² is 2, the minimum number of covalent bonds linking the coordination atoms at M¹-O¹—N⁶-M² and M²-O¹—N⁵-M¹ is 3, and the minimum number of covalent bonds linking the coordination atoms at M¹-N⁵—N⁶-M² is 4.

The multinuclear complex having this combination of coordination atoms is a multinuclear complex having a coordination geometry with M¹ and M² in close proximity, and such a multinuclear complex is preferred for increased catalytic activity.

The ligand L in the multinuclear complex of the invention has a group represented by general formula (1) and/or (2) above, and a condensate can be obtained by condensation reaction via these groups. The condensate can also serve as a highly thermostable catalyst.

The multinuclear complex can also be converted to a co-condensate by co-condensation with a monomer capable of condensation reaction with one or more different groups represented by general formula (1) or (2), and such co-condensates can also serve as highly stable catalysts. The co-condensation is accomplished by condensation of at least one of the aforementioned multinuclear complexes with at least one such monomer. Also, a plurality of monomers may be combined for co-condensation with various multinuclear complex ratios and monomer ratios. The monomer used in this case may be any of various compounds such as silane compounds, metal alkoxide compounds and metal hydroxides.

As examples of silane compounds there may be mentioned tetraalkoxysilanes such as tetramethoxysilane, tetraethoxysilane, tetra-n-propoxysilane and tetra-iso-propoxysilane; tetraaryloxysilanes such as tetraphenoxysilane; alkyltrialkoxysilanes such as methyltrimethoxysilane, methyltriethoxysilane, methyltri-n-propoxysilane, methyltri-iso-propoxysilane, ethyltrimethoxysilane and ethyltriethoxysilane; alkenyltrialkoxysilanes such as vinyltrimethoxysilane and vinyltriethoxysilane; alkenyltriacetoxysiloxysilanes such as vinyltriacetoxysilane; aryltrialkoxysilanes such as phenyltrimethoxysilane, phenyltriethoxysilane, 1,4-bis(triethoxysilyl)benzene and 4,4′-bis(triethoxysilyl)biphenyl; dialkyldialkoxysilanes such as dimethyldimethoxysilane, dimethyldiethoxysilane, diethyldimethoxysilane and diethyldiethoxysilane; diaryldialkoxysilanes such as diphenyldimethoxysilane and diphenyldiethoxysilane; trialkylmonoalkoxysilanes such as trimethylmethoxysilane, trimethylethoxysilane, triethylmethoxysilane and triethylethoxysilane; triarylmonoalkoxysilanes such as triphenylmethoxysilane and triphenylethoxysilane; (meth)acryloxyalkyltrialkoxysilanes such as γ-methacryloxypropyltrimethoxysilane and γ-methacryloxypropyltriethoxysilane; (meth)acryloxyalkylalkyldialkoxysilanes such as γ-methacryloxypropylmethyldiethoxysilane; cycloalkylalkyltrialkoxysilanes such as 3,4-epoxycyclohexylethyltrimethoxysilane; glycidoxyalkyltrialkoxysilanes such as γ-glycidoxypropyltrimethoxysilane and γ-glycidoxypropyltriethoxysilane; glycidoxyalkylalkyldialkoxysilanes such as γ-glycidoxypropylmethyldiethoxysilane; halogenoalkyltrialkoxysilanes such as γ-chloropropyltrimethoxysilane; mercaptoalkyltrialkoxysilanes such as γ-mercaptopropyltrimethoxysilane; mercaptoalkylalkyldialkoxysilanes such as γ-mercaptopropylmethyldimethoxysilane; aminoalkyltrialkoxysilanes such as γ-aminopropyltrimethoxysilane, γ-aminopropyltriethoxysilane and γ-(2-aminoethyl)aminopropyltrimethoxysilane; aminoalkylalkyldialkoxysilanes such as γ-(2-aminoethyl)aminopropylmethyldimethoxysilane; aminoalkyltrialkoxysilanes such as N-phenyl-γ-aminopropyltrimethoxysilane; perfluoralkyltrialkoxysilanes such as perfluorooctylethyltriethoxysilane, perfluorooctylethyltrimethoxysilane, 3,3,3-trifluoropropyltriethoxysilane, 3,3,3-trifluoropropyltrimethoxysilane and 3,3,3-trifluorobutyltriethoxysilane; perfluoralkoxyalkyltrialkoxysilanes such as 3-perfluoroethoxypropyltrimethoxysilane; and bis(3,3,3-trifluoropropyl)diethoxysilane.

Partial condensates of organosiloxanes may also be mentioned as silane compounds, and examples thereof include partial condensates of tetraalkoxysilanes and partial condensates of alkylalkoxysilanes.

The partial condensates of tetraalkoxysilanes may be products that are commonly available on the market, examples of which include “Ethylsilicate 40”, “Methylsilicate 51” and “Methylsilicate 56” by Colcoat Co., Ltd., and “Ethylsilicate 40” and “Ethylsilicate 45” by Tama Chemicals Co., Ltd. As examples of commercially available partial condensates of alkylalkoxysilanes there may be mentioned “KC89”, “KR500” and “KR213” by Shin-Etsu Chemical Co., Ltd., “DC3037” and “SR2402” by Toray/Dow Corning, Inc. and “TSR145” by Toshiba Silicone.

As examples of metal alkoxides there may be mentioned niobium pentaethoxide, magnesium diisopropoxide, aluminum triisopropoxide, tri-n-butoxyaluminum, zinc dipropoxide, tetra-iso-propoxytitanium, tetra-n-butoxytitanium, barium diethoxide, barium diisopropoxide, triethoxyborane, tetra-n-propoxyzirconium, tetra-iso-propoxyzirconium, tetra-n-butoxyzirconium, lanthanum tripropoxide, yttrium tripropoxide and lead diisopropoxide.

As examples of metal hydroxides there may be mentioned compounds obtained by partial hydrolysis of the aforementioned silane compounds or metal alkoxides.

As preferred condensable monomers there may be mentioned tetramethoxysilane, tetraethoxysilane, tetra-n-propoxysilane, tetra-iso-propoxysilane, tetraphenoxysilane, methyltrimethoxysilane, methyltriethoxysilane, methyltri-n-propoxysilane, methyltri-iso-propoxysilane, ethyltrimethoxysilane, ethyltriethoxysilane, vinyltrimethoxysilane, vinyltriethoxysilane, phenyltrimethoxysilane, phenyltriethoxysilane, 1,4-bis(triethoxysilyl)benzene and 4,4′-bis(triethoxysilyl)biphenyl, more preferably tetramethoxysilane, tetraethoxysilane, tetra-n-propoxysilane, tetra-iso-propoxysilane, vinyltrimethoxysilane, vinyltriethoxysilane, phenyltrimethoxysilane, phenyltriethoxysilane, 1,4-bis(triethoxysilyl)benzene and 4,4′-bis(triethoxysilyl)biphenyl, even more preferably tetramethoxysilane, tetraethoxysilane, tetra-n-propoxysilane, tetra-iso-propoxysilane, vinyltrimethoxysilane, vinyltriethoxysilane and aryltrialkoxysilanes such as 1,4-bis(triethoxysilyl)benzene and 4,4′-bis(triethoxysilyl)biphenyl, and most preferably aryltrialkoxysilanes such as 4-bis(triethoxysilyl)benzene and 4,4′-bis(triethoxysilyl)biphenyl.

The condensation or co-condensation reaction may be carried out without a solvent, or it can be carried out using a reaction solvent. It will normally be carried out in the presence of a reaction solvent, and the reaction system may be either a homogeneous or heterogeneous system. The reaction solvent is not particularly restricted, and as examples there may be mentioned water, tetrahydrofuran, ether, 1,2-dimethoxyethane, acetonitrile, benzonitrile, acetone, methanol, ethanol, isopropanol, ethylene glycol, 2-methoxyethanol, 1-methyl-2-pyrrolidinone, dimethylformamide, dimethyl sulfoxide, acetic acid, hexane, pentane, benzene, toluene, xylene, dichloromethane, chloroform and carbon tetrachloride. A single solvent may be used alone or a combination of two or more thereof may be used in combination. Water can most effectively promote the (co)condensation reaction because it has an effect of converting Si—R²⁰ in the group represented by general formula (1) or Si—R⁴⁰ in the group represented by general formula (2) to a silanol group (Si—OH) by hydrolysis, and the silanol group increases the reactivity for (co)condensation. The reaction solvent is therefore preferably a water-containing reaction solvent, and specifically there may be mentioned water-tetrahydrofuran, water-acetonitrile, water-acetone, water-methanol, water-ethanol, water-isopropanol, water-ethylene glycol, water-(2-methoxyethanol), water-(1-methyl-2-pyrrolidinone), water-dimethylformamide, water-dimethyl sulfoxide and water-acetic acid. More preferred are water-tetrahydrofuran, water-acetonitrile, water-methanol, water-ethanol, water-isopropanol, water-ethylene glycol and water-(2-methoxyethanol), and especially preferred are water-tetrahydrofuran, water-acetonitrile, water-methanol and water-ethanol.

As a general procedure for the condensation or co-condensation step, first the multinuclear complex is dispersed in a solvent. If necessary, the condensation catalyst or additives mentioned below may also be included. For co-condensation, the monomer mentioned above is also added. The mixture containing the multinuclear complex is stirred for condensation reaction. The solvent and volatilizing components are then removed from the mixture by drying to obtain a condensate or co-condensate. The drying removal may be carried out under reduced pressure.

The condensation or co-condensation reaction step and the step of drying removal of the solvent and volatilizing components (drying step) may be carried out under heated conditions. Such heating can accelerate the reaction step and drying step. The upper limit for the heating temperature is preferably below 300° C., more preferably below 250° C., even more preferably below 200° C. and most preferably below 150° C. The lower limit for the heating temperature may be appropriately optimized depending on the type of reaction solvent used, in a range that does not cause problems by decomposition of the multinuclear complex used and the (co)condensate produced. A more preferred heating temperature is 20° C. or higher and below 300° C., even more preferably 40° C. or higher and below 250° C. and most preferably 60° C. or higher and below 150° C., with appropriate adjustment to a temperature range that does not degrade the structure of the multinuclear complex used. The phrase “degrade the structure of the multinuclear complex” means breaking of all of the coordination bonds of the multinuclear complex.

The condensation reaction or co-condensation reaction step, and if necessary the solvent and volatilizing component drying step, may be carried out under reduced pressure or under pressure.

As examples of condensation catalysts to be used in the condensation reaction or co-condensation reaction there may be mentioned the following acidic compounds and basic compounds. As examples of acidic compounds there may be mentioned hydrofluoric acid, hydrochloric acid, hydrobromic acid, hydroiodic acid, sulfuric acid, nitric acid, formic acid, acetic acid, phosphoric acid, trifluoroacetic acid, methanesulfonic acid, p-toluenesulfonic acid and trifluoromethanesulfonic acid.

As examples of basic compounds there may be mentioned lithium hydroxide, sodium hydroxide, potassium hydroxide, rubidium hydroxide, cesium hydroxide, calcium hydroxide, strontium hydroxide, barium hydroxide, tetrabutylammonium hydroxide, tetraethylammonium hydroxide, lithium carbonate, sodium carbonate, potassium carbonate, rubidium carbonate, cesium carbonate, lithium phosphate, sodium phosphate, potassium phosphate, rubidium phosphate, cesium phosphate, ammonia, lithium methoxide, sodium methoxide, potassium ethoxide, potassium butoxide, triethylamine and pyridine.

The aforementioned basic compounds are preferred as catalysts, among which lithium hydroxide, sodium hydroxide, potassium hydroxide, rubidium hydroxide, cesium hydroxide, calcium hydroxide, strontium hydroxide, barium hydroxide, tetrabutylammonium hydroxide, tetraethylammonium hydroxide, ammonia, triethylamine and pyridine are more preferred, lithium hydroxide, sodium hydroxide, potassium hydroxide, rubidium hydroxide, cesium hydroxide, tetrabutylammonium hydroxide, tetraethylammonium hydroxide and ammonia are even more preferred, and lithium hydroxide, sodium hydroxide, potassium hydroxide and ammonia are most preferred.

As examples of additives to be used for the condensation reaction or co-condensation reaction, there may be mentioned a variety of different kinds of additives including surfactants such as nonionic surfactants, anionic surfactants, cationic surfactants, fluorine-containing surfactants and the like, leveling agents, thickeners, organosilanes such as “HAS1” and “HSA6” by Colcoat Co., Ltd. or “DC6-2230” and “SH6018” by Toray/Dow Corning, Inc., curing aids such as hydrolysates of metal alkoxides, adhesion aids such as various coupling agents, coloring agents such as dyes and pigments, extender pigments and the like, colloidal fine particles such as colloidal silica and colloidal alumina, metal oxide sols such as titanium oxide, tin oxide, ATO, ITO and the like, ultraviolet absorbers, antioxidants, antifungal agents, peroxides, diazo compounds and the like.

The multinuclear complex obtained in the manner described above, the condensate obtained by condensation of the multinuclear complex or the co-condensate obtained from the multinuclear complex and another condensable monomer, is a heterogeneous catalyst exhibiting the unique catalytic power of the multinuclear complex itself, and may be suitably used as a redox catalyst, for example.

The present invention is not in any way limited to the preferred modes described above.

### EXAMPLES

The present invention will now be explained in greater detail through the following examples, with the understanding that these examples are not limitative on the invention.

### Production Example 1

### Ligand Synthesis

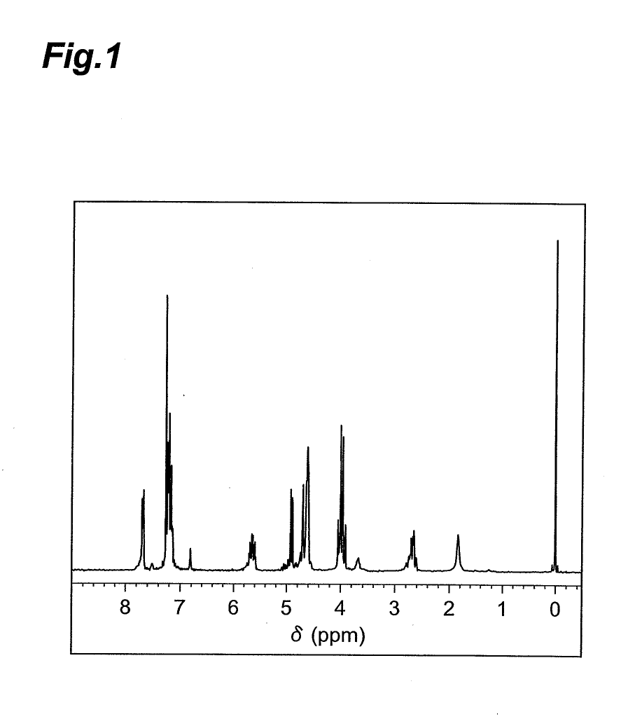

A compound represented by the following formula (7) (hereinafter referred to as “bbpr-allyl ligand”) was synthesized according to the HL-Et ligand described in J. Am. Chem. Soc. 1984, 106, p. 4765-4772. Specifically, 2-hydroxy-1,3-diaminopropanetetraacetic acid (10.0 g, 32.0 mmol) was reacted with o-diaminobenzene (21.1 g, 194 mmol) and purified. Next, 5.00 g (8.20 mmol) of the obtained purified product was allylated with allyl chloride (3.25 g, 42.4 mmol) to obtain 4.51 g of bbpr-allyl ligand (71% yield). The obtained bbpr-allyl ligand was subjected to ¹H-NMR (0.05% (v/v) TMS CDCl₃ solution) measurement, and a peak at 4-6 ppm confirmed successful introduction of allyl groups. The ¹H-NMR spectrum of the obtained bbpr-allyl ligand is shown in FIG. 1.

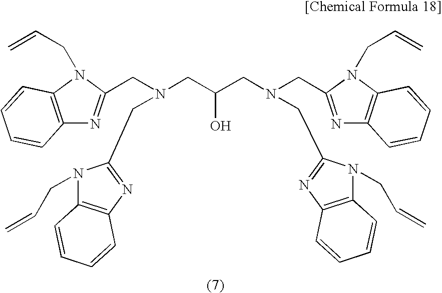

### Example 1

### Production of Multinuclear Complex

A multinuclear complex (hereinafter referred to as “Mn-(bbpr-allyl)-OTf”) was synthesized according to the method described in J. Am. Chem. Soc. 1994, 116, pp. 891-897. Specifically, the bbpr-allyl ligand (1.63 g, 2.11 mmol) obtained in Production Example 1 was reacted with manganese acetate tetrahydrate (1.18 g, 4.80 mmol) and sodium triflate (0.826 g, 4.80 mmol) to obtain 2.09 g of Mn-(bbpr-allyl)-OTf (80% yield).

Elemental analysis: Calcd for C₅₁H₅₂F₆Mn2N₁₀O₉S₂: C, 49.52; H, 4.24; N, 11.32. Found: C, 49.55; H, 4.37; N, 11.71.

[Hydrosilylation Reaction]

The Mn-(bbpr-allyl)-OTf (100 mg, 0.0808 mmol) was dissolved in ethanol (30 mL), and then triethoxysilane (60 μL, 3.20×10⁻⁴ mol) and a Karstedt catalyst xylene solution (0.1 mol/L, 3 μL) were added and the mixture was stirred at 65° C. for 4 days. After the reaction, the solvent was removed under reduced pressure to obtain 113 mg of the multinuclear complex “Mn-(bbpr-SiOR)—OTf” represented by the following formula (8). Disappearance of allyl groups and introduction of silyl groups in the obtained multinuclear complex was confirmed by the IR spectrum and Raman spectrum. The reaction procedures were conducted under a nitrogen atmosphere using a glove box and Schlenk line, and the ethanol and triethoxysilane used had been dried over magnesium followed by distillation. Elemental analysis of the obtained multinuclear complex gave the following results: C, 40.50; H, 5.20; N, 7.07; Mn, 6.01.

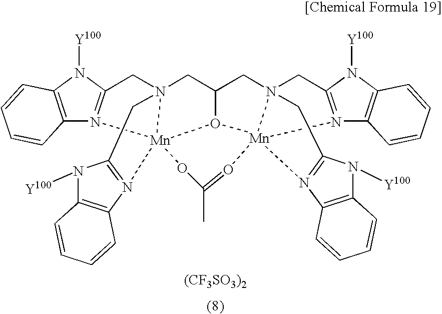

Here, Y¹⁰⁰ represents any of the following triethoxysilyl group-containing groups, and the four Y¹⁰⁰ groups may be the same or different.

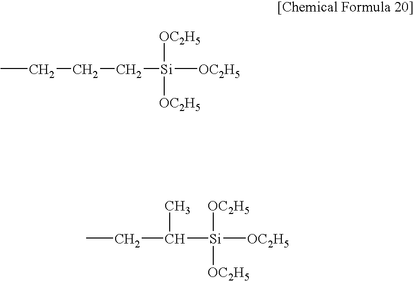

### Example 2

### Co-Condensate Production 1

The multinuclear complex “Mn-(bbpr-SiOR)—OTf” (99.2 mg) obtained in Example 1 was dissolved in ethanol (800 μL), and then 1,4-bis(triethoxysilyl)benzene (210 μL, 5.29×10⁻⁴ mol) was added, and a mixture including water (70 μL) and a sodium hydroxide ethanol solution (1.01 mol/L, 760 μL) was further added. The produced gel was washed, subjected to centrifugal separation and then vacuum dried to obtain 120 mg of “Mn-(bbpr-silox)-OTf/DSB”, a co-condensate of the Mn-(bbpr-SiOR)—OTf and 1,4-bis(triethoxysilyl)benzene. The ethanol that was used had been treated by drying distillation. Analysis of the manganese content of the obtained co-condensate (ICP optical emission spectrometry) showed a value of 4.00 wt %.

### Example 3

### Co-Condensate Production 2

The multinuclear complex “Mn-(bbpr-SiOR)—OTf” (39.7 mg) obtained in Example 1 was dissolved in ethanol (400 μL), and then 1,4-bis(triethoxysilyl)benzene (84 μL, 2.11×10⁻⁴ mol) was added, and a mixture comprising water (122 μL) and a lithium hydroxide ethanol solution (0.581 mol/L, 172 μL) was further added. The produced gel was dried at 80° C. for 2 hours and then pulverized with a mortar, washed with an ethanol/water mixture (volume ratio=1/1) and vacuum dried at 80° C. for 2 hours to obtain 40.9 mg of “Mn-(bbpr-silox)-OTf/DSB”, a co-condensate of Mn-(bbpr-SiOR)—OTf and 1,4-bis(triethoxysilyl)benzene. The ethanol that was used had been treated by drying distillation. Analysis of the manganese content of the obtained co-condensate (ICP optical emission spectrometry) showed a value of 3.72 wt %.

### Example 4

### Peroxide Decomposition Test 1 Using Co-Condensate

The co-condensate “Mn-(bbpr-silox)-OTf/DSB” obtained in Example 2 was used as a heterogeneous catalyst for a peroxide decomposition test. First, 11.59 mg (8.44 μmol (per metal atom)) of “Mn-(bbpr-silox)-OTf/DSB” was placed in a 25 mL two-neck flask, and a solution of poly(sodium 4-styrenesulfonate) (product of Aldrich Co., weight-average molecular weight: approximately 70,000) dissolved in tartaric acid/sodium tartrate buffer (prepared from 0.20 mol/L aqueous tartaric acid solution and 0.10 mol/L aqueous sodium tartrate solution, pH 4.0) to a polymer concentration of 21.1 mg/mL was added (1.00 mL), after which ethylene glycol (1.00 mL) was added and the mixture was stirred.

A septum was fitted on one of the necks of the two-neck flask containing this solution, while a gas buret was connected to the other neck. The flask contents were stirred at 80° C. for 5 minutes as heat treatment before the reaction, and a hydrogen peroxide aqueous solution (11.4 mol/L, 0.20 mL (2.28 mmol)) was added with a syringe for a peroxide decomposition test at 80° C. for 20 minutes. The generated oxygen was measured through the gas buret to quantify the amount of decomposed hydrogen peroxide. The reaction mixture was then diluted with a water/acetonitrile mixture (volume ratio=7/3) to a solution volume of 10.0 mL, and the solution was filtered with a syringe filter. The filtrate was measured by GPC under the conditions indicated below, and the weight-average molecular weight of the poly(sodium 4-styrenesulfonate) after the test was determined by conversion using a calibration curve for standard (polyethylene oxide)s. The weight-average molecular weight of the poly(sodium 4-styrenesulfonate) before the test was measured by GPC under the same conditions and compared therewith to examine the degree of low molecularization of the poly(sodium 4-styrenesulfonate) by free radicals produced by hydrogen peroxide via single electron transfer, and amount of the generated free radicals was calculated. The results of measuring the weight-average molecular weight are shown in Table 1.

[GPC Analysis Conditions]

Device: L2000 (trade name of Hitachi Corp.)
Column: TSKgel α-M (trade name of Tosoh Corp., 13 μm, 7.8 mmφ×30 cm)
Column temperature: 40° C.
Mobile phase: 50 mmol/L aqueous ammonium acetate solution/CH₃CN (volume ratio=7/3)
Flow rate: 0.6 mL/min

### Detector: RI

Injection rate: 50 μL

[Measurement of Peroxide Decomposition Quantity]

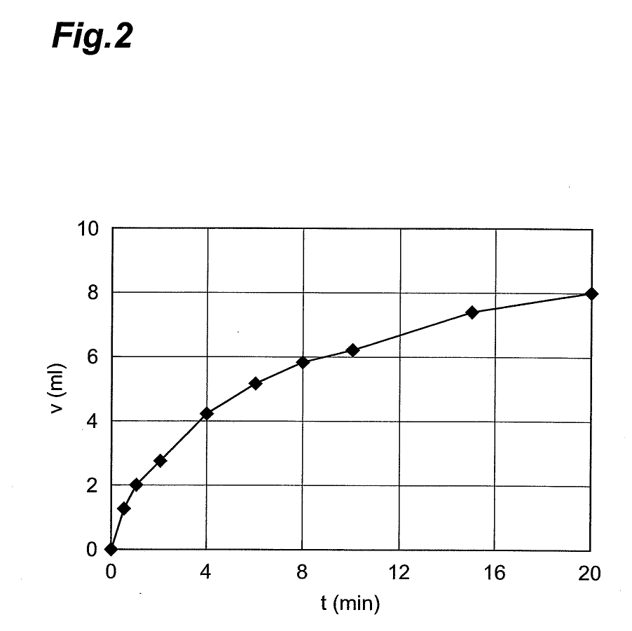

The actual volume value, obtained by measuring the generated oxygen by the gas buret in the peroxide decomposition test described above, was determined by the following equation, assuming conditions of 0° C., 101,325 Pa (760 mmHg) in consideration of steam pressure, for quantitation of the hydrogen peroxide decomposition. FIG. 2 shows the change in converted value for the generated oxygen volume with time (where t is the elapsed time).

$$V = \frac{273 v ( P - p )}{760 ( 273 + t )} \qquad [ Equation 1 ]$$

In this equation, P represents the atmospheric pressure (mmHg), p represents the water vapor pressure (mmHg), t represents the temperature (° C.), v represents the measured volume of generated gas (mL), and V represents the gas volume (mL) at 0° C., 101325 Pa (760 mmHg).

### Example 5

### Peroxide Decomposition Test 2 Using Co-Condensate

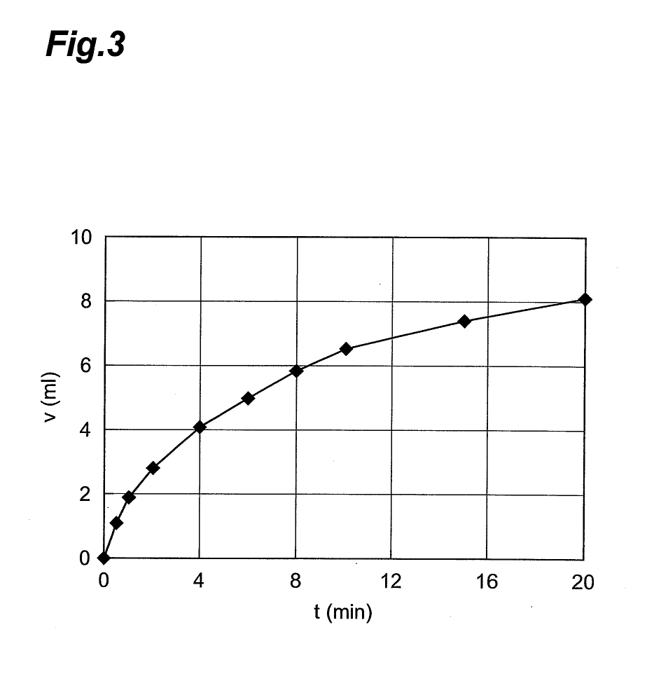

First, 12.41 mg (8.41 μmol (per metal atom)) of the co-condensate “Mn-(bbpr-silox)-OTf/DSB” obtained in Example 3 was used as a heterogeneous catalyst for a process in the same manner as in Example 4, and the co-condensate was subjected to a peroxide decomposition test. The results of measuring the weight-average molecular weight of the poly(sodium 4-styrenesulfonate) after the test are shown in Table 1, and a graph of time-dependent change in the generated oxygen converted by the method described above is shown in FIG. 3.

TABLE 1

| | Weight-average molecular weight |
|---|---|
| Poly(sodium 4-styrenesulfonate) | 1.0 × 10⁵ |
| before test | |
| Example 4 | 9.9 × 10⁴ |
| Example 5 | 9.4 × 10⁴ |

As shown in FIG. 2 and FIG. 3, oxygen was produced by decomposition of hydrogen peroxide as time elapsed, thus demonstrating that the co-condensates obtained in Examples 2 and 3, as heterogeneous catalysts, exhibit hydrogen peroxide-decomposing catalytic activity. As clearly shown in Table 1, the weight-average molecular weights of the poly(sodium 4-styrenesulfonate) compounds in the reaction systems of Example 4 and Example 5 were values that were essentially equivalent to the poly(sodium 4-styrenesulfonate) compounds before each test. This demonstrated that the co-condensates obtained in Examples 2 and 3 had decomposed hydrogen peroxide with minimal generation of free radicals, and that they have high catalyst selectivity as heterogeneous catalysts.

### Example 6

### Co-Condensate Production 3

The multinuclear complex “Mn-(bbpr-SiOR)—OTf” (60.0 mg) obtained in Example 1 was dissolved in ethanol (12.0 mL), and then 4,4′-bis(triethoxysilyl)biphenyl (145 μL, 3.17×10⁻⁴ mol) and aqueous sodium hydroxide (4.86 mg of sodium hydroxide dissolved in 142 μL of water) was added and the mixture was stirred for 7 days. The produced gel was washed, subjected to centrifugal separation, washed with ethanol and water in that order and then dried at 80° C. to obtain 57.0 mg of “Mn-(bbpr-silox)-OTf/DSbP”, a co-condensate of Mn-(bbpr-SiOR)—OTf and 4,4′-bis(triethoxysilyl)biphenyl.

### Production Example 2

### Ligand Synthesis

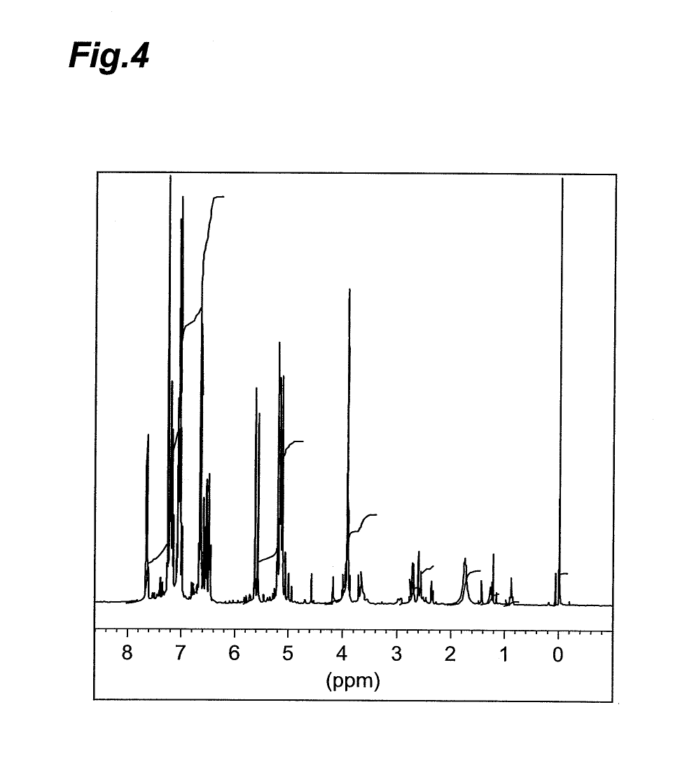

The bbpr-CH₂St ligand represented by the following formula (9) was obtained at an 85% yield in the same manner as Production Example 1, except that 4-chloromethylstyrene was used instead of allyl chloride. The obtained bbpr-CH₂St ligand was subjected to ¹H-NMR (0.05% (v/v) TMS CDCl₃ solution) measurement, and a peak at 5-8 ppm confirmed successful introduction of —CH₂St groups. The ¹H-NMR spectrum of the obtained bbpr-CH₂St ligand is shown in FIG. 4.

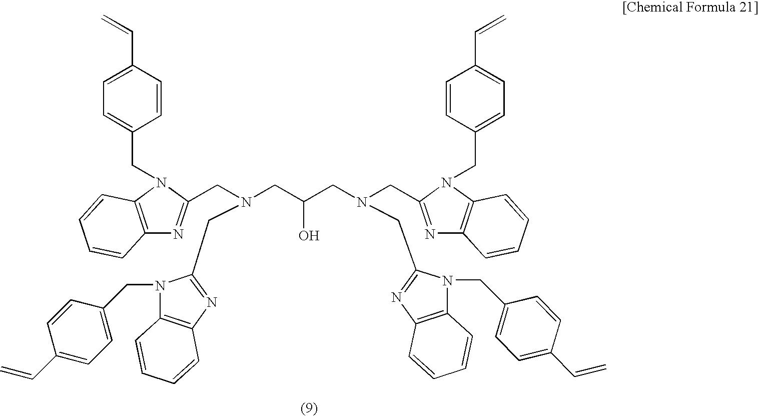

### Production Example 3

### Synthesis of Multinuclear Complex Precursor

After placing p-vinylbenzoic acid (10.1 g, 67.5 mmol) and an aqueous sodium hydroxide solution (10.2 g, 64.1 mmol) in a flask, 140 mL of water was added thereto, the mixture was stirred to dissolution and the insoluble components were filtered out to prepare an aqueous sodium p-vinylbenzoate solution. In a separate flask there were placed manganese sulfate pentahydrate (7.74 g, 32.1 mmol) and 50 ml of water, and the mixture was stirred to dissolution. After adding the aqueous sodium p-vinylbenzoate solution thereto, the mixture was stirred at room temperature for 2 hours. The produced precipitate was filtered and washed with water and ether in that order, and then dried under reduced pressure to obtain a white powder of manganese p-vinylbenzoate.4H₂O in an amount of 5.87 g (13.9 mmol, 43% yield).

Elemental analysis: Calcd for C₁₈H₂₂MnO₈: C, 51.32; H, 5.26. Found: C, 51.63; H, 5.16.

### Example 7

### Production of Multinuclear Complex

After placing the ligand bbpr-CH₂St (400 mg, 0.372 mmol) obtained in Production Example 2 and diisopropylethylamine (43.2 mg, 0.335 mmol) in a flask, 54 mL of tetrahydrofuran was added and the mixture was stirred to dissolution. Next, the aforementioned manganese p-vinylbenzoate 4H₂O (313 mg, 0.744 mmol) was added and the mixture was stirred at room temperature for 2 hours. The reaction mixture was concentrated under reduced pressure, methanol was added, and the produced precipitate was filtered and washed with water and ether in that order and then dried under reduced pressure to obtain 122 mg of the “Mn-vb-(bbpr-CH₂St)-vb” represented by the following formula (10), as a beige powder.

ESI MS [M-(p-vinylbenzoic acid anion)]⁺=1477.4

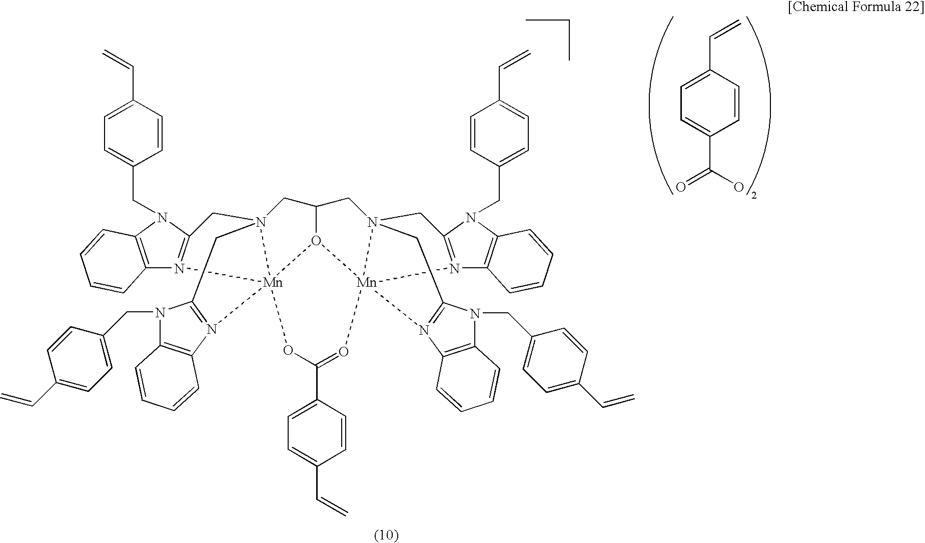

[Hydrosilylation Reaction]

The obtained Mn-vb-(bbpr-CH₂St)-vb (100 mg) was dissolved in tetrahydrofuran (30 mL), and then triethoxysilane (60 μL, 3.20×10⁻⁴ mmol) and a Karstedt catalyst xylene solution (0.1 mol/L, 3 μL) were added and the mixture was stirred at room temperature for 7 days. After the reaction, the solvent was removed under reduced pressure to obtain the multinuclear complex Mn-vb-(bbpr-CH₂StSiOR)-vbSiOR (180 mg) represented by the following formula (11), as the reaction product of Mn-vb-(bbpr-CH₂St)-vb and triethoxysilane. Introduction of silyl groups into the obtained multinuclear complex was confirmed based on the IR spectrum.

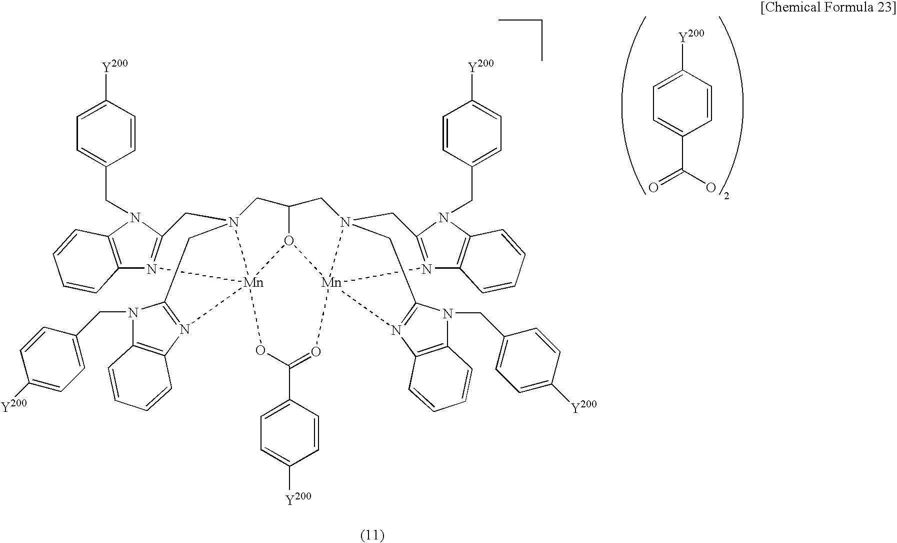

Here, Y²⁰⁰ represents any of the following triethoxysilyl group-containing groups, and the seven Y²⁰⁰ groups may be the same or different.

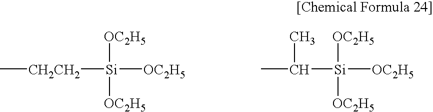

### Example 8

### Production of Multinuclear Complex

After placing bbpr-CH₂St (1.46 g, 1.36 mmol), diisopropylethylamine (0.160 g, 1.24 mmol) and cobalt acetate 4H₂O (0.686 mg, 2.75 mmol) in a flask, dimethyl sulfoxide (50 mL) was added for dissolution. The solution was stirred for 1 hour, and then sodium tetraphenylborate (0.941 mg, 5.50 mmol) was added and the mixture was stirred for 30 minutes. Water was added to the reaction mixture, and the produced precipitate was filtered and washed with water and ether in that order and vacuum dried to obtain 2.48 g of “Co-(bbpr-CH₂St)-BPh₄” represented by the following formula (12) (62% yield).

ESI MS [M-(BPh₄)]⁺=1570.6

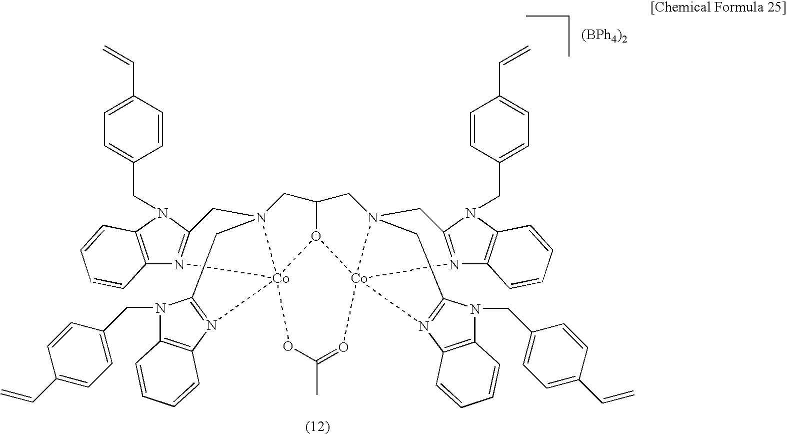

[Hydrosilylation Reaction]

The obtained Co-(bbpr-CH₂St)-BPh₄ (105 mg) was dissolved in tetrahydrofuran (30 mL), and then triethoxysilane (96 μL, 5.12×10⁻⁴ mol) and a Karstedt catalyst xylene solution (0.1 mol/L, 3 μL) were added and the mixture was stirred at room temperature for 7 days. After the reaction, the solvent was removed under reduced pressure to obtain the multinuclear complex Co-(bbpr-CH₂StSiOR)—BPh₄ (220 mg) represented by the following formula (13), as the reaction product of Co-(bbpr-CH₂St)-BPh₄ and triethoxysilane. Introduction of silyl groups into the obtained multinuclear complex was confirmed based on the IR spectrum.

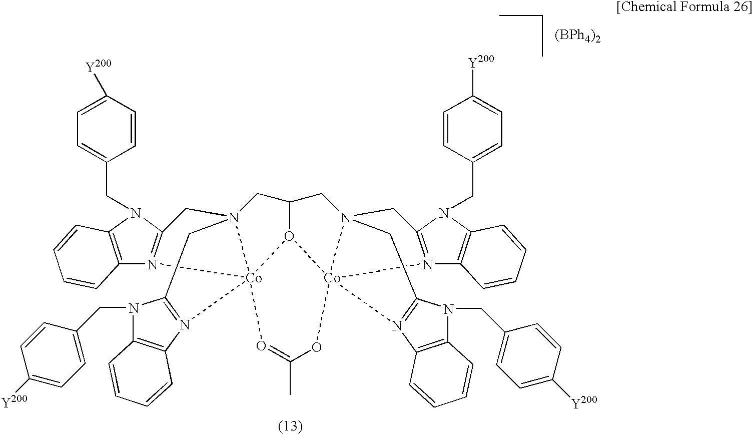

Here, Y²⁰⁰ has the same definition as above and the four Y²⁰⁰ groups may be the same or different.

### Example 9

### Production of Multinuclear Complex

The compound “Ni-(bbpr-CH₂St)-BPh₄” (2.55 g) represented by the following formula (14) was obtained in the same manner as Example 8, except that nickel acetate.4H₂O (0.687 mg, 2.75 mmol) was used instead of cobalt acetate.4H₂O (0.686 mg, 2.75 mmol) (62% yield).

ESI MS [M-(BPh₄)]⁺=1568.5

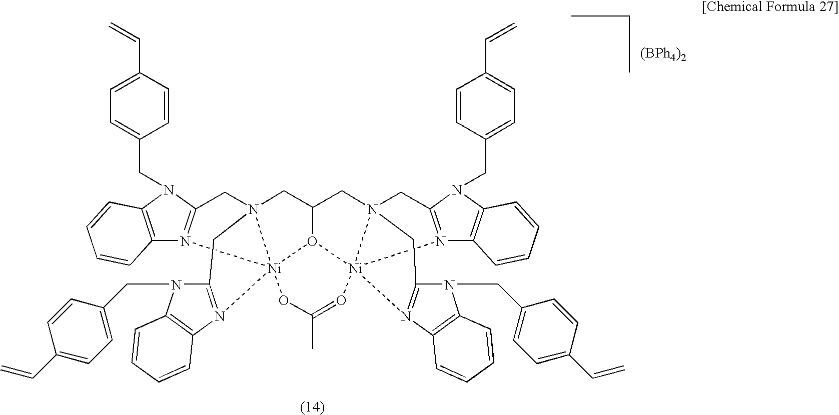

[Hydrosilylation Reaction]

The multinuclear complex Ni-(bbpr-CH₂StSiOR)—BPh₄ (200 mg) represented by the following formula (15) was then obtained as a reaction product of Ni-(bbpr-CH₂St)-BPh₄ and triethoxysilane, in the same manner as Example 8 except for using Ni-(bbpr-CH₂St)-BPh₄ (105 mg) instead of Co-(bbpr-CH₂St)-BPh₄. Introduction of silyl groups into the obtained multinuclear complex was confirmed based on the IR spectrum.

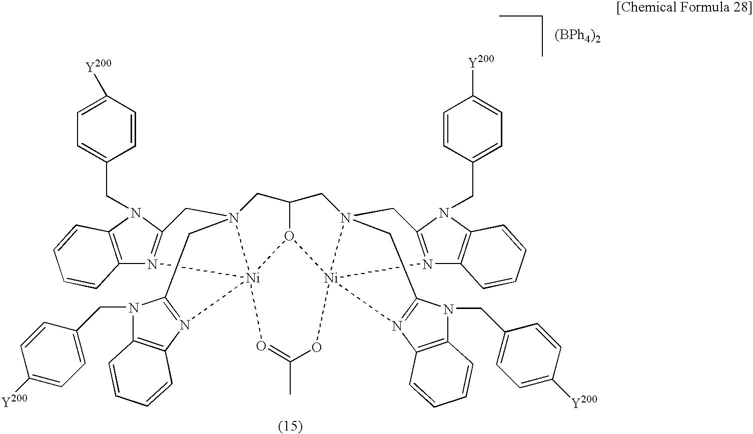

Here, Y²⁰⁰ has the same definition as above and the four Y²⁰⁰ groups may be the same or different.

### Example 10

### Production of Multinuclear Complex

The compound “Cu-(bbpr-CH₂St)-BPh₄” (2.39 g) represented by the following formula (16) was obtained in the same manner as Example 8, except that copper(II) acetate.H₂O (0.549 mg, 2.75 mmol) was used instead of cobalt acetate.4H₂O (0.686 mg, 2.75 mmol) (78% yield).

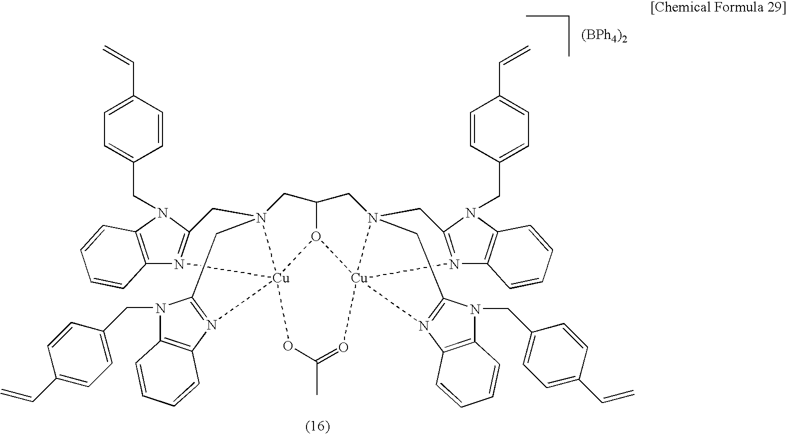

[Hydrosilylation Reaction]

The multinuclear complex Cu-(bbpr-CH₂StSiOR)—BPh₄ (178 mg) represented by the following formula (17) was then obtained as a reaction product of Cu-(bbpr-CH₂St)-BPh₄ and triethoxysilane, in the same manner as the [Hydrosilylation reaction] of Example 8 except for using Cu-(bbpr-CH₂St)-BPh₄ (101 mg) instead of Co-(bbpr-CH₂St)-BPh₄. Introduction of silyl groups into the obtained multinuclear complex was confirmed based on the IR spectrum.

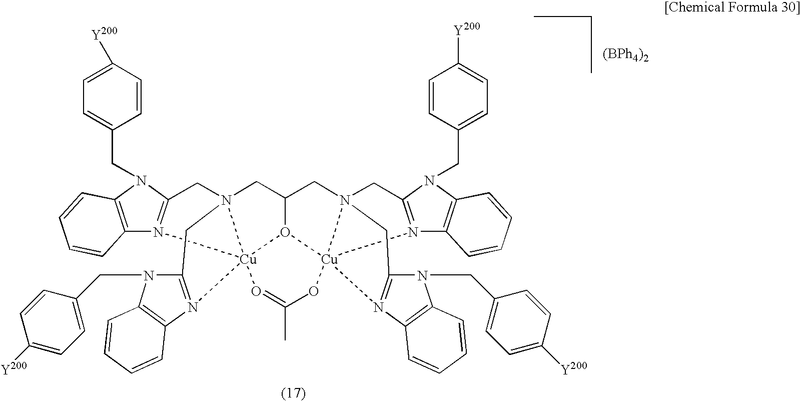

Here, Y²⁰⁰ has the same definition as above and the four Y²⁰⁰ groups may be the same or different.

### Example 11

### Production of Multinuclear Complex

The compound “Fe-(bbpr-CH₂St)-BPh₄” (2.77 g) represented by the following formula (18) was obtained in the same manner as Example 8, except that iron(II) chloride.4H₂O (0.545 mg, 2.77 mmol) was used instead of cobalt acetate.4H₂O (0.686 mg, 2.75 mmol) (62% yield).

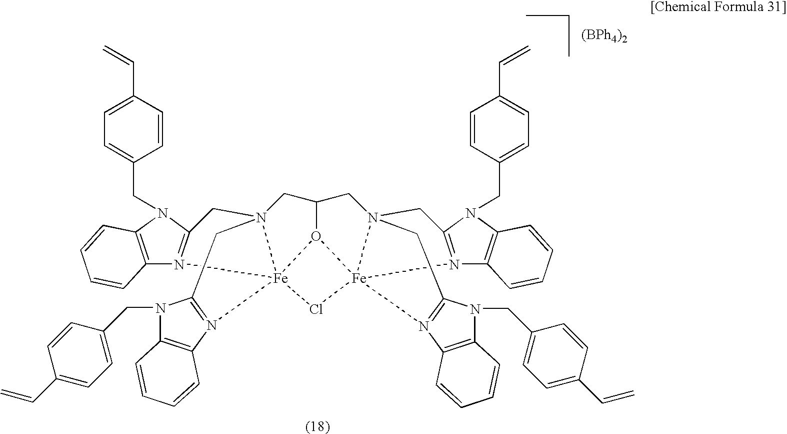

[Hydrosilylation Reaction]

The multinuclear complex Fe-(bbpr-CH₂StSiOR)—BPh₄ (122 mg) represented by the following formula (19) was then obtained as a reaction product of Fe-(bbpr-CH₂St)-BPh₄ and triethoxysilane, in the same manner as Example 8 except for using Fe-(bbpr-CH₂St)-BPh₄ (99.5 mg) instead of Co-(bbpr-CH₂St)-BPh₄. Introduction of silyl groups into the obtained multinuclear complex was confirmed based on the IR spectrum.

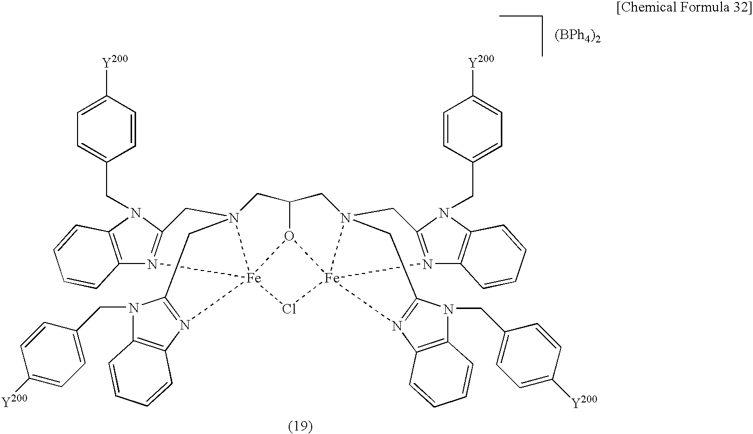

Here, Y²⁰⁰ has the same definition as above and the four Y²⁰⁰ groups may be the same or different.

### Example 12

### Production of Multidentate Ligand

After mixing bbpr-allyl (15 mg) with acetonitrile (40 mL), triethoxysilane (100 μL) and a Karstedt catalyst xylene solution (3 wt %, 1 drop) were added and the mixture was stirred at 65° C. for 4 days. After the reaction, the solvent was removed under reduced pressure to obtain the hydrosilylation product bbpr-allylSiOR containing the following formula (20). The reaction procedures were conducted under a nitrogen atmosphere using a glove box and Schlenk line, and the acetonitrile and triethoxysilane used had been dried over calcium hydride and magnesium followed by distillation. The obtained product was identified by its ¹H and ¹³C-NMR spectra. In ¹H-NMR, the disappearance of a signal (4.5-6.0 ppm) derived from the allyl groups of the starting compound and the appearance of a signal (1.5 ppm, 0.6 ppm) derived from Y¹⁰⁰ of the product were observed. In ¹³C-NMR as well, the disappearance of a signal (132 ppm, 116 ppm) derived from the allyl groups of the starting compound and the appearance of a signal (14 ppm, 11 ppm, 8 ppm, 3 ppm) derived from Y¹⁰⁰ of the product were observed.

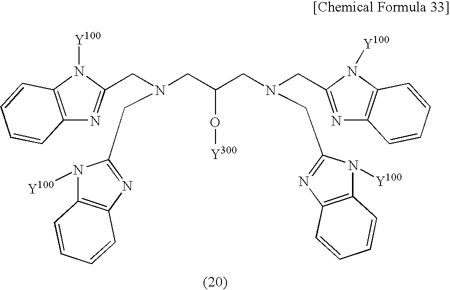

Here, Y¹⁰⁰ represents any of the following triethoxysilyl group-containing groups, and the four Y¹⁰⁰ groups may be the same or different.

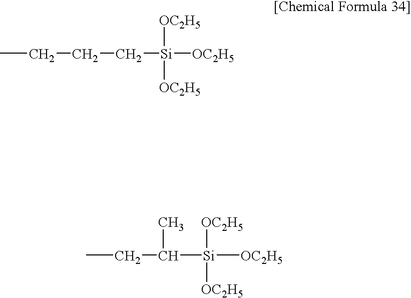

Y³⁰⁰ represents hydrogen or a triethoxysilyl or diethoxysilyl group as shown below.

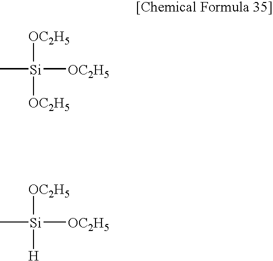

### INDUSTRIAL APPLICABILITY

The multinuclear complex, the condensate obtained by condensation of the multinuclear complex and the co-condensate obtained by co-condensation of the multinuclear complex, according to the invention, are useful as redox catalysts. In particular, when any of these are used as a peroxide decomposition catalyst, co-condensation with the condensate allows decomposition to water and oxygen to be accomplished while minimizing generation of free radicals, and yields a heterogeneous catalyst that is insoluble in solvents, unlike hitherto disclosed multinuclear complex catalysts. Such a heterogeneous catalyst facilitates catalyst separation and recovery from the reaction system and conjugation with materials, and can be used as an antidegradant for polyelectrolyte fuel cells and hydroelectrolysis devices or as an antioxidant for medical and agricultural chemicals and food products.

1. A multinuclear complex comprising a plurality of metal atoms and a ligand L coordinating to the metal atoms, and satisfying the following conditions (i), (ii), (iii) and (iv):
(i) the ligand L has a monovalent group represented by the following general formula (1) and/or a divalent group represented by the following general formula (2), 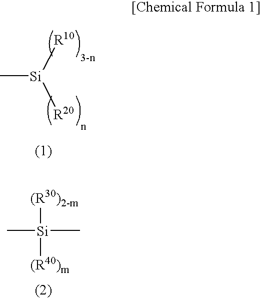
wherein R¹⁰ and R³⁰ represent an optionally substituted C1-10 alkyl or optionally substituted C₆₋₁₀ aryl group; when more than one R¹⁰ and R³⁰ are bonded to the same Si, they may be the same or different; R²⁰ and R⁴⁰ each independently represent hydrogen, hydroxyl, optionally substituted C1-10 alkoxy, optionally substituted C₆₋₁₀ aryloxy, optionally substituted C2-10 acyloxy or —O—P(O)(OR⁷⁰)₂ (where R⁷⁰ represents hydrogen or a C1-10 alkyl or C₆₋₁₀ aryl group), and when more than one R²⁰ and R⁴⁰ are bonded to the same Si, they may be the same or different; the letter n represents 1, 2 or 3, and m represents 1 or 2;
(ii) the ligand L has at least 5 coordination atoms bonding to the metal atoms,
(iii) at least one of the coordination atoms bonds to two of the metal atoms, or the minimum number of covalent bonds between any two selected coordination atoms is 1-5; and
(iv) the ligand L is soluble in the solvent.

2. A multinuclear complex according to claim 1, wherein the coordination atom is a nitrogen atom, oxygen atom, phosphorus atom or sulfur atom.

3. A multinuclear complex according to claim 1, wherein at least one of the coordination atoms is a nitrogen atom that forms a bond represented by —C═N—.

4. A multinuclear complex according to claim 1, wherein the total number of metal atoms is no greater than 8.

5. A multinuclear complex according to claim 1, wherein the metal atom is a transition metal atom of the first series of transition elements.

6. A multinuclear complex according to claim 1, wherein the number of ligands L is 1, and the number of metal atoms is 2.

7. A multinuclear complex according to claim 1, wherein the molecular weight is no greater than 6000.

8. A compound represented by the following general formula (3): 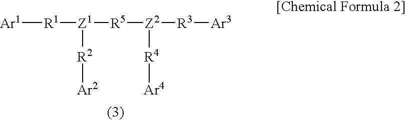
wherein Ar¹, Ar², Ar³ and Ar⁴ each independently represent an aromatic heterocyclic group, R¹, R², R³, R⁴ and R⁵ each independently represent a divalent group, and Z¹ and Z² each independently represent a nitrogen atom or trivalent group; at least one of Ar¹, Ar², Ar³, Ar⁴, R¹, R², R³, R⁴ and R⁵ has a monovalent group represented by general formula (1) and/or a divalent group represented by the following general formula (2): 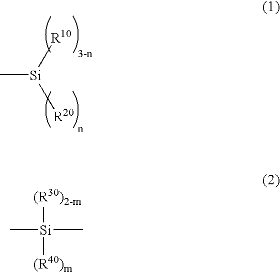
wherein R¹⁰ and R³⁰ represent an optionally substituted C1-10 alkyl or optionally substituted C6-10 aryl group: when more than one R¹⁰ and R³⁰ are bonded to the same Si, they may be the same or different; R²⁰ and R⁴⁰ each independently represent hydrogen, hydroxyl, optionally substituted C1-10 alkoxy, optionally substituted C6-10 aryloxy, optionally substituted C2-10 acyloxy or —O—P(O)(OR⁷⁰)₂ (where R⁷⁰ represents hydrogen or a C1-10 alkyl or C₆₋₁₀ aryl group), and when more than one R²⁰ and R⁴⁰ are bonded to the same Si, they may be the same or different; and the letter n represents 1, 2 or 3, and m represents 1 or 2.

9. A compound according to claim 8 which is represented by the following general formula (4a) or (5a): 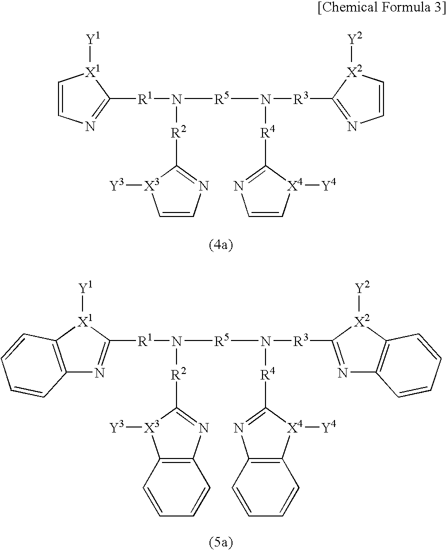
wherein R¹, R², R³, R⁴ and R⁵ in (4a) and (5a) have the same definitions as above; X¹, X², X³ and X⁴ each independently represent a nitrogen atom or CH group, Y¹, Y², Y³ and Y⁴ each independently represent hydrogen, a C1-50 alkyl group or a C2-60 aromatic group, or a group having the structure represented by general formula (1) above; and at least one of Y¹, Y², Y³ and Y⁴ is a group containing a group represented by general formula (1) above.

10. A compound according to claim 9 which is represented by the following general formula (4b) or (5b): 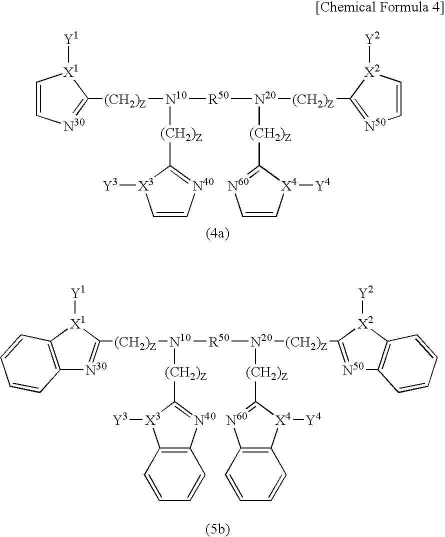
wherein X¹, X², X³, X⁴, Y¹, Y², Y³ and Y⁴ in (4b) and (5b) have the same definitions as above; at least one of Y¹, Y², Y³ and Y⁴ is a group containing a group represented by general formula (1) above, and Z represents 1 or 2; and R⁵⁰ represents a divalent group with 2-14 covalent bonds linking N10 and N²⁰.

11. A compound according to claim 10 which is represented by the following general formula (4c) or (5c): 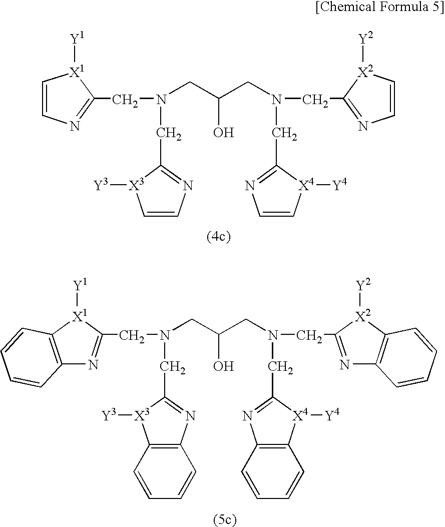
wherein X¹, X², X³, X⁴, Y¹, Y², Y³ and Y⁴ in (4c) and (5c) have the same definitions as above, and at least one of Y¹, Y², Y³ and Y⁴ is a group containing a group represented by general formula (1) above.

12. A multinuclear complex according to claim 1, which contains a compound according to claim 8 as the ligand L.

13. A condensate obtained by condensation of a multinuclear complex according to claim 1.

14. A condensate according to claim 13, wherein the reaction temperature for condensation is below 150° C.

15. A co-condensate obtained by co-condensation of one or more multinuclear complexes according to claim 1 with a monomer capable of co-condensation with the multinuclear complex.

16. A co-condensate according to claim 15, wherein the reaction temperature for co-condensation is below 150° C.

17. A redox catalyst comprising a multinuclear complex according to claim 1.

18. A redox catalyst comprising a condensate according to claim 13.

19. A redox catalyst comprising a co-condensate according to claim 15.
